(19) United States
(12) Patent Application Publication
(10) Pub. No.: US 2026/0348912 A1
(43) Pub. Date: Nov. 5, 2026

(54) METHOD OF SENSING AN ANALYTE USING MACHINE LEARNING

(71) Applicant: EYE3CONCEPTS INC., Oakville (CA)

(72) Inventors: Seyyedeh Hoda MOZAFFARI, North Aurora, IL (US); Greter Amelia ORTEGA RODRIGUEZ, Hamilton (CA); Herlys VILTRES COBAS, Hamilton (CA); Syed Rahin AHMED, Hamilton (CA); Seshasai SRINIVASAN, Brampton (CA); Amin Reza RAJABZADEH, Burlington (CA)

(21) Appl. No.: 19/104,316

(22) PCT Filed: Aug. 17, 2023
(86) PCT No.: PCT/CA2023/051089

§ 371 Date: Feb. 17, 2025

Related U.S. Application Data
(60) Provisional application No. 63/399,156, filed on Aug. 18, 2022.

(51) Int. Cl. G01N 27/48; G01N 27/327

(52) U.S. Cl. CPC G01N 27/48; G01N 27/3271

(57) ABSTRACT

There is provided a method of sensing an analyte in a sample by electrochemical detection. The sample is received on a sample receiving region of an electrochemical sensor, the sample receiving region being in fluid communication with a sensing electrode of the sensor. An electric potential scan is applied in a target range of electric potentials to the sensing electrode to induce an electrochemical reaction with the analyte. An electrical signal is measured from the sensing electrode while the electric potential is applied. The electrical signal is inputted into a processing device having at least one machine learning algorithm operating therein. The processing device executes the at least one machine learning algorithm to determine from the electrical signal a presence or an absence of the analyte in the sample.

### CROSS-REFERENCE TO RELATED APPLICATIONS

This patent application claims priority on U.S. Patent Application No. 63/399,156, filed on Aug. 18, 2022, the entire contents of which are incorporated herein by reference.

### TECHNICAL FIELD

This disclosure relates generally to methods of sensing analytes using machine learning.

### BACKGROUND

Many electrochemical sensors have been developed to detect different analytes in biological samples or biologically derived samples. Examples of electrochemical sensors include microfluidic chips and test strips. The advantages of microfluidic chips include fast assay time, reduced volume of reagents and samples, increased accuracy, ease of use and portability. Most biological samples, such as body fluids (e.g. oral fluids) are a complex matrix that make the detection of analytes difficult due to many potential sources of interference in the sample and because of natural variance between different subjects the biological sample is obtained from. In the exemplary case of saliva, the viscosity of human saliva is approximately 1.30 times higher than water, thereby affecting the analyte's diffusion and the reaction rates on the electrodes of electrochemical sensors. In addition, saliva has various natural or adulterant electroactive components that may interfere with the analyte electrochemical performance. Moreover, the pH, conductivity, and the protein-chemical-solid compositions of saliva, among others, change over time and vary from subject to subject. Furthermore, there are inherent batch-to-batch discrepancies during the manufacturing and modification of electrodes for electrochemical sensors. This leads to additional challenges in obtaining reproducible and accurate results on a commercial scale. Accordingly, improvements in the accuracy and reproducibility of methods of sensing analytes are desired.

### SUMMARY

In one aspect, there is provided a method of sensing an analyte in a sample by electrochemical detection, the method comprising:
receiving the sample on a sample receiving region of an electrochemical sensor, the sample receiving region being in fluid communication with a sensing electrode of the sensor;
applying an electric potential scan in a target range of electric potentials to the sensing electrode to induce an electrochemical reaction with the analyte;
measuring an electrical signal from the sensing electrode while the electric potential is applied;
inputting the electrical signal into a processing device having at least one machine learning algorithm operating therein; and
executing, by the processing device, the at least one machine learning algorithm to determine from the electrical signal a presence or an absence of the analyte in the sample.

In some embodiments, the sensing electrode has a plurality of sensor analytes associated therewith. In some embodiments, the electrochemical reaction is an oxidation, a reduction, or an enzymatic reaction. In some embodiments, the electrical signal is an electric current. In some embodiments, the sample comprises a body fluid, cells from a subject or a biomolecule from the subject. In some embodiments, the sample comprises one or more of oral fluid, sputum, urine, tears, blood, plasma, nasal fluid, sweat, cerebral spinal fluid, suspended cells, and feces. In some embodiments, the electric potential is applied using a voltammetric technique. In some embodiments, the voltammetric technique is selected from square wave voltammetry, cyclic voltammetry, linear sweep voltammetry, and differential pulse voltammetry. In some embodiments, the voltammetric technique is square wave voltammetry. In some embodiments, the target range of electric potential is from 0 to 5 V. In some embodiments, the at least one machine learning algorithm is executed by the processing device to determine the presence or the absence of the analyte in the sample comprising determining a range of values of a concentration of the analyte in the sample. In some embodiments, the at least one machine learning algorithm is executed by the processing device to determine the presence or the absence of the analyte in the sample comprising determining a single value of a concentration of the analyte in the sample. In some embodiments, the at least one machine learning algorithm is executed by the processing device to determine the presence or the absence of the analyte in the sample comprising determining whether the concentration of the analyte in the sample is above or below a predetermined concentration threshold. In some embodiments, the at least one machine learning algorithm is configured to decrease noise present in the electrical signal for determining the presence or the absence of the analyte in the sample, the noise resulting from at least one of subject-to-subject variations in the sample, discrepancies between batches of the sensing electrode, and analog compound interference in the sample. In some embodiments, the at least one machine learning algorithm is trained with at least one statistical feature of the electrical signal, the at least one statistical feature comprising at least one of a maximum, a minimum, a distance between the maximum and the minimum, a mean, a variance, a skewness, and a kurtosis. In some embodiments, the at least one machine learning algorithm is trained with an entirety of the electrical signal. In some embodiments, the method further comprising reducing a dimensionality of the electrical signal prior to executing the at least one machine learning algorithm. In some embodiments, the dimensionality of the electrical signal is reduced using one of principal component analysis (PCA), locally linear embedding (LLE), multidimensional scaling (MDS), t-distributed stochastic neighbor embedding (t-SNE), and linear discriminant analysis (LDA). In some embodiments, the at least one machine learning algorithm is a supervised machine learning algorithm or an unsupervised machine learning algorithm. In some embodiments, the at least one machine learning algorithm is configured to perform at least one of a regression analysis and a classification task to determine the concentration of the analyte from the electrical signal. In some embodiments, the at least one machine learning algorithm is configured to perform the classification task using one of logistic regression, soft Regression, decision Tree, random forest (RF), and an artificial neural network (ANN). In some embodiments, the at least one machine learning algorithm is configured to perform the regression analysis using one of linear regression, gradient descent, polynomial regression, regularized linear model, ridge regression, lasso regression, and support vector machine (SVM). In some embodiments, the at least one machine learning algorithm uses XGBoost (eXtreme Gradient Boosting). In some embodiments, the at least one machine learning algorithm comprises a plurality of different machine learning algorithms combined into an ensemble machine learning model. In some embodiments, the analyte is a metabolite, a drug of abuse, or a hormone.

Many further features and combinations thereof concerning the present improvements will appear to those skilled in the art following a reading of the instant disclosure.

### BRIEF DESCRIPTION OF THE DRAWINGS

FIG. 1 is a flow chart of an example method for sensing an analyte in a sample by electrochemical detection.

FIG. 2 is a schematic graph of a support vector machine (SVM) regression, which can also be referred to as a support vector regression (SVR).

FIG. 3 is a schematic representation of a neuron of an artificial neural network (ANN).

FIG. 4 is a block diagram of a detection system for detecting the sample analyte.

FIG. 5A is a graph showing square wave voltammetry (SWV) signals during the THC deposition of different modified electrodes (m-Zensor, m-Z) with 0, 2, or 5 ng/mL of tetrahydrocannabinol (THC).

FIG. 5B is a graph showing the raw data of three m-Zensor and one pristine (P-Z) per THC concentration 0, 2, and 5 ng/mL.

FIG. 5C is a graph showing an example of the subtraction of the signals for the samples (THC 0, 2, and 5 ng/mL) recovered with m-Z-THC minus the signal obtained with pristine Zensor (FIG. 5A and FIG. 5B).

FIG. 6A is a bar graph showing the sensor electrochemical performance for three saliva samples (S1, S2, S3) with THC collected with OFCD-100 swab and with no filtration.

FIG. 6B is a bar graph showing the sensor electrochemical performance for three saliva samples (S1, S2, S3) with THC collected with OFCD-100 swab and after being filtered with glass wool. The THCi was 100 ng.

FIG. 7 is a bar graph showing the sensor electrochemical performance using different THC initial deposition amounts (Dep) of 100, 130, and 150 ng and testing synthetic saliva (SS) and five biological saliva samples (S5-S8) with THC 0, 2, and 5 ng/mL (respectively left to right for each of 100, 130, and 150 ng) collected and filtered with OFCD-100 swab/glass wool.

FIG. 8A is a bar graph showing sensor electrochemical performance using different a batch labeled “Batch 2” of Zensor electrodes and THC initial deposition amount of 130 ng. Four real saliva samples (S9-S12) with THC 0, 2, and 5 ng/mL collected and filtered with OFCD-100 swab/glass wool were tested.

FIG. 8B is a bar graph showing sensor electrochemical performance using a different batch labeled “Batch 3” of Zensor electrodes and THC initial deposition amount of 130 ng. Four real saliva samples (S9-S12) with THC 0, 2, and 5 ng/mL collected and filtered with OFCD-100 swab/glass wool were tested.

FIG. 9 is a bar graph showing sensor electrochemical performance using different potentiostats (mono-potentiostat (P) and multichannel potentiostat (MP) and THC initial deposition amount of 130 ng. Six biological saliva samples (S13-S18) with THC 0, 2, and 5 ng/mL collected and filtered with OFCD-100 swab/glass wool were tested.

FIG. 10A is a schematic representation of the oxidation of the THC and cannabidiol (CBD) molecules.

FIG. 10B is a square wave voltammetric (SVM) response of THC-based (m-Z-THC, 130 ng) and CBD-based (m-Z-CBD, 100 ng) sensors in phosphate buffered saline (PBS).

FIG. 10C is a graph showing the current in function of potential for the electrodes pristine Zensor (P-Z), m-Z-THC (130 ng) and m-Z-CBD (100 ng).

FIG. 11A is a graph showing the SWV response of electrochemical sensors modified with THC (m-Z-THC) for 0, 2, and 5 ng/mL THC detection in the presence of interferences (CBD 0, 10, and 50 ng/mL) in human saliva.

FIG. 11B is a graph showing the SWV response of electrochemical sensors modified with CBD (m-Z-CBD) for 0, 2, and 5 ng/mL CBD detection in the presence of interferences (THC 0, 10, and 50 ng/mL) in human saliva.

FIG. 11C is a bar graph showing the SWV response of electrochemical sensors modified with THC (m-Z-THC) for 0, 2, and 5 ng/mL THC detection in the presence of interferences (CBD 0, 10, and 50 ng/mL) in six human saliva samples (S1-S6).

FIG. 11D is a bar graph showing the SWV response of electrochemical sensors modified with CBD (m-Z-CBD) for 0, 2, and 5 ng/mL CBD detection in the presence of interferences (THC 0, 10, and 50 ng/mL) in six human saliva samples (S1-S6).

FIG. 12A is a histogram showing the training of saliva samples containing THC 0 ng/mL in the presence of CBD, and using m-Z-THC sensor.

FIG. 12B is a histogram showing the results of saliva samples containing THC 0 ng/mL in the presence of CBD, and using m-Z-THC sensor, following the training in FIG. 12A.

FIG. 12C is a histogram showing the training of saliva samples containing THC 2 ng/mL in the presence of CBD, and using m-Z-THC sensor.

FIG. 12D is a histogram showing the results of saliva samples containing THC 2 ng/mL in the presence of CBD, and using m-Z-THC sensor, following the training in FIG. 12C.

FIG. 12E is a histogram showing the training of saliva samples containing THC 5 ng/mL in the presence of CBD, and using m-Z-THC sensor.

FIG. 12F is a histogram showing the results of saliva samples containing THC 5 ng/mL in the presence of CBD, and using m-Z-THC sensor, following the training in FIG. 12E.

FIG. 13A is a graph showing the feature importance as determined by the mean decrease in impurity (MDI).

FIG. 13B is a graph showing the feature importance as determined by the mean decrease in accuracy (MDA).

FIG. 14A is a graph showing the accuracy in function of the maximum depth of the training set for a RF model evaluated with Gini impurity (for a number of trees of 5, 10, 20, 40, 80, and 160).

FIG. 14B is a graph showing the accuracy in function of the maximum depth of the testing set for a RF model evaluated with Gini impurity (for 5, 10, 20, 40, 80, and 160 trees).

FIG. 15A is a graph showing the accuracy in function of the maximum depth of the training set for a RF model evaluated with entropy (for 5, 10, 20, 40, 80, and 160 trees).

FIG. 15B is a graph showing the accuracy in function of the maximum depth of the testing set for a RF model evaluated with entropy (for 5, 10, 20, 40, 80, and 160 trees).

FIG. 16 is a graph showing the accuracy in function of the number of minimum samples for the RF model.

FIG. 17A is a graph showing the accuracy in function of the number of principal components (PC) in the training set for different Kernel functions using the SVM model.

FIG. 17B is a graph showing the accuracy in function of the number of principal components (PC) in the testing set for different Kernel functions using the SVM model.

FIG. 18A is a graph showing the training accuracy in function of the batch size for ANN design 1 training set.

FIG. 18B is a graph showing the training accuracy in function of the batch size for ANN design 1 testing set.

FIG. 18C is a graph showing the training accuracy in function of the batch size for ANN design 2 training set.

FIG. 18D is a graph showing the training accuracy in function of the batch size for ANN design 2 testing set.

FIG. 18E is a graph showing the training accuracy in function of the batch size for ANN design 3 training set.

FIG. 18F is a graph showing the training accuracy in function of the batch size for ANN design 3 testing set.

FIG. 18G is a graph showing the training accuracy in function of the batch size for ANN design 4 training set.

FIG. 18H is a graph showing the training accuracy in function of the batch size for ANN design 4 testing set.

FIG. 18I is a graph showing the training accuracy in function of the batch size for ANN design 5 training set.

FIG. 18J is a graph showing the training accuracy in function of the batch size for ANN design 5 testing set.

FIG. 19 is a graph showing the computation time in function of the batch size for ANN design 3.

### DETAILED DESCRIPTION

Machine learning (ML) techniques are at the intersection of statistics and computer science where computers can learn from past data without explicit programming. The major applications of ML algorithms are classification, regression, and anomaly detection tasks.

There is provided a method of sensing the presence or absence of an analyte in a sample by electrochemical detection using an electrochemical sensor and at least one machine learning (ML) algorithm. The present method has the advantage of reducing, limiting or preferably removing the possible interferences during the sensing assay when determining the presence or absence of the analyte, or when determining a concentration range or concentration value of the analyte. The interference can be caused by sample to sample variation between different subjects. When the interference is not accounted for when determining the presence or absence of analyte, and more particularly when determining a concentration range or a concentration value of the analyte, a significant loss of accuracy in the determination would occur. Moreover, additional sources of interference, error or noise include batch-to-batch variation of commercial electrodes used in electrochemical sensors, the type of electric reader, the saliva collection method and pre-treatment (if any).

One of the objectives of the present disclosure is to account for substances that can be an interference in the electrical signal. These interfering substances can generate similar electrical signals, which can result in the wrong analyte detection or in the masking of the electrical signal of the analyte. ML algorithms have been successfully demonstrated herein to be able to fulfill this objective. Additional ways to reduce signal interference, for example, are to perform a sample pre-treatment procedure, such as a solid-phase extraction, a chromatography, or other separation methods to separate out sources of interferences (e.g. molecules or cells). Another example of these methodologies is the use of molecularly imprinted nanoparticles (nanoMIPs) such as sequestering (masking) agents, which can help suppress the interfering signal. Furthermore, electrode modifications with macrocyclic compounds can reduce interference due to their anti-interference capacity against coexisting ions or molecules.

The sample may contain electroactive agents that can interfere with the detection of the sample analyte (i.e. act as interference). The electroactive agents can be, without limitations, an organic component (such as for example a polymer, an acid, a base, charged molecules and the like), an inorganic component (such as, for example a salt which can be, without limitation, NaCl, NH₄Cl, NaH₂PO₄, KCl, Na₃Cit, MgCl₂, Na₂CO₃, CaCl₂ or a combination thereof) and/or a biological component (such as, for example, a protein such as enzyme, the protein can be, without limitation, albumin, lysozyme, mucin or a combination thereof).

ML algorithms offer solutions to a complex and large-size data system involving problems that traditionally required tedious hand-tuning rules and tasks with fluctuating environments. ML refers to computational techniques that are learned from past experiences (i.e. data) to create logical and precise prediction algorithms. The data used in these learning algorithms influence the success of ML models; hence ML is the intersection of data analysis and statistics with computer programming. The ML algorithm of the present disclosure can be supervised or unsupervised.

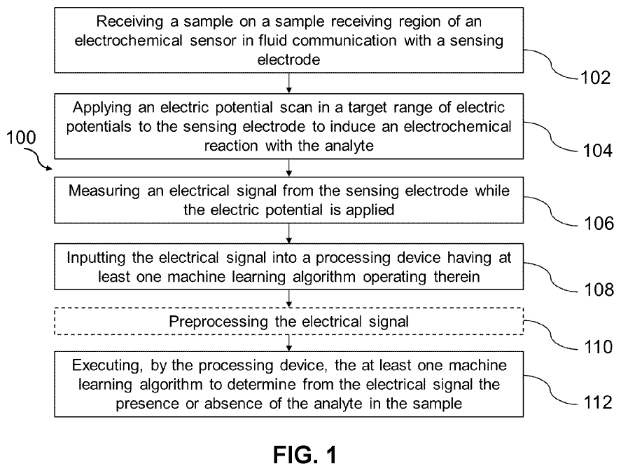

Making reference to FIG. 1, there is provided a method 100 of sensing an analyte in a sample by electrochemical detection. At step 102, the method comprises receiving a sample on the sensing electrode (by providing, for example, the sample to the sample receiving region of the sensor in fluid communication with the sensing electrode). Briefly, an electric potential scan is applied 104 in a target range of potentials to the sensing electrode to induce an electrochemical reaction with the analyte. The electric signal is measured 106 while the electric potential is applied 104. The electric signal is inputted 108 into a processing device having at least one ML algorithm operating therein. The electric signal is optionally preprocessed 110. The processing device executes 112 the at least one ML algorithm to determine from the electric signal the presence or absence of the analyte in the sample.

Returning to step 102, in some embodiments, the sample is a biological sample (such as a bodily fluid, cells from a subject or a biomolecule from the subject) that is obtainable non-invasively (e.g., an ex vivo bodily fluid). For example, the sample can be an oral fluid sample such as saliva or sputum, a lavage, or an epithelial swab of a subject's tissue (e.g. nasal or oral swab). Other examples of non-invasive bodily fluids include, but are not limited to, urine, sweat, tears, nasal fluid, suspended cells, and feces. In some embodiments, the sample may be a bodily fluid that is obtainable invasively such as blood, plasma, suspended cells or cerebral spinal fluid. In one embodiment, the sample has been obtained from an animal, such as a mammalian animal (a human or horse for example), a plant or a microorganism. As such, the method of detection described herein can include a step of obtaining a sample from a subject (which can be an animal, a plant or a microbe).

In some embodiments, the sample can be used without prior treatment and just be received on the sample receiving region of the sensor after collection. In other embodiments, the sample can be treated before being received on the sample receiving region of the sensor. In such embodiments, a treated sample will be received on the sample receiving region of the sensor. A treated sample comprises a component of a sample and refers to a sample that has been treated before the detection process. The treatment can include, without limitation, the removal of at least one component of the sample (such as solid residues, proteins, polynucleotides, charged entities, and the like), the dilution of the sample, the freezing of the sample or the heat-treatment of the sample. Preferably, the treatment is performed in a way so as to preserve, as much as possible, the integrity (and especially the electrochemical state) of the sample analyte.

In a specific embodiment, the sample comprises saliva or a saliva component suspected of comprising the sample analyte. The saliva sample can be provided without any treatment steps and received on the sample receiving region of the sensor. The saliva sample can optionally be treated before being received on the sample receiving region. In one embodiment, the saliva sample can be submitted to a dilution, a filtration, a centrifugation, a precipitation, a pH adjustment or a combination thereof. In a further embodiment, the saliva sample is filtered prior to being received in the sample receiving region of the sensor. In a specific example, a partial filtration can be performed during the collection of saliva with different material, such as, for example swabs made of cotton, cellulose, or synthetic fibers. In another example, the filtration can be performed with a filter having a pore size of between about 0.1 to 0.5 μm or between about 0.1 to 3 μm including filters having diameters between 10 to 30 mm. The filtering membrane can be, but is not limited to, GHP (hydrophilic propylene), hydrophobic PTFE (Polytetrafluoroethylene), PES (Polyethersulfone), hydrophobic PVDF (Polyvinylidene fluoride), nylon, glass wool (treated or untreated), hydrophobic PTFE (Polytetrafluoroethylene), hydrophilic PTFE or any combination thereof. In one embodiment, the pH of the saliva sample can be adjusted by adding a base, an acid or a buffer. In another embodiment, the saliva sample can be dissolved in an alcoholic solvent (such as methanol or ethanol), a buffer (such as phosphate buffer saline (PBS)), or a combination of both. In one embodiment the ratio of dilution of the saliva sample is between 1:10 to 10:1, between 1:5 to 5:1, between 1.2:1 to 1:1.2, or about 1:1.

When the sample intended to be used is a solid material (e.g. cells, viruses or a cellular component), it can be first dissolved or suspended in a solvent (such as water or a buffer) prior to being placed on the sample receiving region of the electrochemical sensor. The obtained solution or suspension can be subjected to any of the sample treatment steps described herein.

When the sample intended to be used is saliva, the sensor can be placed in the oral cavity of the subject to facilitate the contact of the subject's bodily fluid (saliva in this embodiment) with the sample receiving region of the sensor. In an embodiment, the sensor can be placed in the vicinity of the tongue or mandible of the subject and can even, in some further embodiments, be placed in contact with the subject's tongue or mandible (to gather, for example, submandibular and/or sublingual saliva). Alternatively or in combination, the sample can be obtained from a collecting means and then received on the sample receiving region of the sensor. The collecting means may include collecting saliva by placing a porous filter media into the subject's oral cavity, which absorbs saliva found in the oral cavity, and subsequently expressing the saliva onto the sensor. Alternatively, saliva can be collected by a subject expectorating into a container and subsequently transferring a sufficient volume of saliva onto the sensor. In an embodiment, a sufficient volume of the sample is such that the sample covers the surface of the sensing electrode and optionally the baseline electrode of the electrochemical sensor. In an embodiment, the volume of the sample received in the sample-receiving region is between about 50 μL to about 1 mL. These values may vary depending on practical implementations.

At step 104, the method 100 provides applying an electric potential scan in a target range of electric potentials to the sensing electrode to induce an electrochemical reaction with the analyte. The electrochemical reaction can be an oxidation reaction, a reduction reaction or an enzymatic reaction that has an electrochemical component (e.g. transfer of electrons or H⁺). In some embodiments, applying the electric potential scan also causes a portion of the reacted sample analyte (e.g. oxidized or reduced sample) to associate with the surface of the sensing electrode. The target range of electric potentials can vary with the different analytes. In some embodiments, the target range of electric potentials is from 0 to 5 V, from 0 to 4 V, from 0 to 3 V, or from 0 to 2 V. In some embodiments, the electric potential scan is applied using a voltammetric technique such as square wave voltammetry (SWV), cyclic voltammetry (CV), linear sweep voltammetry (LSV), and differential pulse voltammetry (DPV). In preferred embodiments, the applying 106 of an electric potential scan is preferably applying SWV.

Voltammetry techniques are electroanalytical techniques that can detect and/or quantify an analyte, by measuring a current as an applied electric potential is varied (i.e. electric potential scan). As described above, the voltammetry techniques can be, but are not limited to, cyclic voltammetry (CV), linear sweep voltammetry (LSV), differential pulse voltammetry (DPV), or square wave voltammetry (SWV). CV is performed by cycling the potential of a working electrode (e.g., the sensing and the baseline electrodes) ramped linearly versus time, and measuring the resulting current. LSV measures the current at the working electrodes (e.g., sensing and baseline electrodes) while the potential between the working electrode and a reference electrode is swept linearly overtime. In the DPV technique a potential scan is recovered by imposing potential pulses with a constant amplitude. The differences between the currents registered just before and at the end of the pulse are plotted versus the potential. SWV is a large-amplitude differential technique in which a waveform composed of a symmetrical square wave, superimposed on a base staircase potential, is applied to the working electrodes (e.g., the sensing and the baseline electrodes).

The analyte of the present disclosure can be any analyte detectable by electrochemistry. For example, the analyte can have an oxidizable group, a reducible group or can undergo an electrochemical reaction with a suitable electrochemical enzyme such as hepatic cytochrome P450 or CYP2C9 for THC and alcohol oxidase (AOX) for alcohol detection (e.g. methanol, ethanol). In embodiments where the analyte undergoes an oxidation or a reduction reaction, the analyte may react and associate with the surface of the sensing electrode. In some embodiments, the sensing electrode comprises sensing analytes associated therewith that promote the interactions between the analyte (i.e. analyte in the sample, referred to herein as “sample analyte”) and the surface of the sensing electrode. Such embodiments can provide an improved sensitivity of detection. When the analyte undergoes an electrochemical reaction it may associate or bind with the sensing electrode surface thereby inducing a change in the resistance and/or conductivity and modifying the current based on the concentration or presence of analyte in the sample. Accordingly, in some embodiments, a portion of the oxidized/reduced sample analyte associates with the sensing electrode. The oxidized/reduced sample analytes can be, at least in part, directly associated with the sensing electrode by directly interacting with the surface of the sensing electrode. In some specific embodiments, the oxidized/reduced sample analyte is integrated in a dimer, an oligomer or a polymer of one or more species of the sensor sample analytes (which can be oxidized or reduced) in which at least one monomeric unit is directly associated with the surface of the sensing electrode. In some embodiments, the majority or the totality of the sample analyte is oxidized/reduced during the detection.

In some embodiments, the analyte is a drug of abuse, a metabolite or a hormone. Drugs of abuse include but are not limited to cannabinoids (e.g. THC), benzodiazepines, opiates including natural opioids (morphine, heroin), narcotics (cocaine), semi-synthetic opioids (oxycodone, hydrocodone, oxymorphone, hydromorphone) and synthetic opioids (fentanyl, methadone, tramadol), steroids, alcohols, amphetamines, barbiturates, buprenorphine, methamphetamines, cotinine, phencyclidine (PCP), 3,4-Methylenedioxymethamphetamine (MDMA), hallucinogens (Lysergic acid diethylamide (LSD), Kratom, Psilocybin), ketamine, gamma hydroxybutyrate (GHB), synthetic cannabinoids (K2/spice). The opiate can be, for example, morphine, hydromorphone and buprenorphine as well as metabolites thereof. The neurotransmitter can be, for example, dopamine, serotonin, or metabolites thereof. The hormone can be, without limitation, a steroid hormone such as, for example, estradiol, estrogen, testosterone, or metabolites thereof. In some embodiments, the analyte is a drug of abuse having a chemical group that can be oxidized and/or a chemical group that can be reduced. For example, the analyte may be a tetrahydrocannabinol or cocaine having an oxidizable group. In some embodiments, the analyte is an alcohol (e.g. methanol or ethanol) and the electrochemical reaction is an enzymatic reaction. The present disclosure also contemplates embodiments where the analyte is glucose in which case a glucose oxidase enzyme can be used for the enzymatic reaction. In some embodiments, the electrochemical reaction or the enzymatic reaction is assisted or mediated by antibodies, metal nanoparticles, multiwalled carbon nanotube (MWCNT), aptamers, or molecularly imprinted polymers (MIPs).

In some embodiments, the analyte is a cannabinoid. The cannabinoid can be, for example, Δ⁹-tetrahydrocannabinol (THC), 11-hydroxy-Δ9-tetrahydrocannabinol (11-hydroxy-THC), delta-8-tetrahydrocannabinol (Δ8-THC), 11-nor-9-carboxy-tetrahydrocannabinol (11-nor-9-carboxy-THC), cannabidiol (CBD), cannabinol (CBN), and glucuronic acid conjugated COOH-THC (gluc-COOH-THC), tetrahydrocannabinolic acid (THCA) or metabolites thereof. The opiate can be, for example, morphine, hydromorphone and buprenorphine as well as metabolites thereof. The neurotransmitter can be, for example, dopamine, serotonin, or metabolites thereof. The hormone can be, without limitation, a steroid hormone such as, for example, estradiol, estrogen, testosterone, or metabolites thereof.

At step 106, the method 100 provides measuring 106 an electrical signal from the sensing electrode while the electric potential is applied 104. The electrical signal is measured by an electric device. In some embodiments, the electrical signal is current and the electric device is an ammeter, a multimeter or a resistor. The current may be measured continuously during the electric potential scan or during a portion of the electric potential scan. The electric device can be coupled or connected to a processing device to provide the electric signal as an input to the processing device (step 108). In some embodiments, the electric device is connected to the processing device in the same electric circuit, for example with electric wires. In other embodiments, the electric device can be coupled to the processing device and can provide the electric signal through an electromagnetic wave (Bluetooth, WI-FI and the like).

In some embodiments, the step 106 of measuring the electrical signal may further comprise measuring the electrical signal of a baseline electrode and/or a reference electrode. The baseline electrode is particularly useful in embodiments where the sensing electrode has a plurality of sensor analytes associated therewith. The current measured at the baseline electrode can be used by the ML algorithm to account for the current contribution in the electric signal of the sensor analytes. Embodiments of the sensor with a sensing electrode having the sensor analytes associated therewith is described in details further below. Reference electrodes can be used in some embodiments to help account for interferences in the sample or may contribute to the electrical signal based on the voltammetry technique selected. Accordingly, in some embodiments, the processing device and ML algorithm can receive an electric signal from the sensing electrode and in addition an electric signal from a baseline electrode and/or a reference electrode. In some embodiments, the ML algorithm receives a signal from the working electrode before any samples is deposited therein.

Optionally at step 110, the electrical signal is preprocessed. In some embodiments, the preprocessing 110 includes reducing the dimensionality of the electrical signal using one of principal component analysis (PCA), locally linear embedding (LLE), multidimensional scaling (MDS), t-distributed stochastic neighbor embedding (t-SNE), and linear discriminant analysis (LDA). The preprocessing may include selecting a statistical feature of the electrical signal and/or performing a statistical analysis/processing on the electrical signal. Preprocessing of data of electric signals is particularly relevant during the training of the ML algorithm. In some embodiments, the same preprocessing step is performed on the electrical signal measured as that performed on the training set of the ML algorithm. In some embodiments, feature rescaling is performed as part of the preprocessing 110. Feature rescaling can eliminate the sensitivity of some ML techniques to different scales in the features. These can be linear and non-linear standardization or normalization. The signal can be rescaled using one of the Standard Scaler, Robust Scaler, Min-Max Scaler, and Power Transformer.

When training a ML algorithm, preprocessing can help mitigate poor quality or insufficient quantity of data which are often considered significant challenges of ML techniques. The performance of ML methods may drop with outliers and noise in the training datasets (increased complexity and computational time). Accordingly, preprocessing allows to obtain a higher-quality dataset. Data cleaning and feature scaling are two main parts of the data preparation phase. Managing missing data is the purpose of data cleaning and can be performed by either eliminating the entire feature or data points related to the feature with missing values, as well as estimating the missing values via reasonable techniques. In addition, most ML techniques are sensitive to the scale of numerical attributes. Different rescaling methods can be carried out based on the nature of the ML technique and datasets.

In some embodiments, the at least one ML algorithm is trained with at least one statistical feature of the electrical signal selected from one or more of a maximum, a minimum, a distance between the maximum and the minimum, a mean, a variance, a skewness, and a kurtosis. In alternative embodiments, the at least one ML algorithm is trained with an entirety of the electrical signal. In general, training with the entirety of the electrical signal can provide an increased accuracy compared to selecting a statistical feature of the electrical signal. However, there is a trade-off in training time and in some cases detection assay time when using an entire electrical signal compared to one or more statistical features. The processing and assay time can be faster when only done with one or more statistical features of the electric signal as opposed to the entirety of the electric signal.

At step 112, the processing device executes at least one ML algorithm to determine from the electrical signal the presence or absence of the analyte in the sample. In some embodiments, determining the presence or the absence of the analyte in the sample comprises determining a range of values of a concentration of the analyte in the sample. For example, the output for such embodiments can be a concentration range or a value with a percentage based error (e.g. ±3%, ±5%, 7%, or ±10%) of the sample analyte. In alternative embodiments, determining the presence or the absence of the analyte in the sample comprises determining a single value of a concentration of the analyte in the sample. In other embodiments, determining the presence or absence of the analyte includes determining whether the concentration of the analyte in the sample is above or below a predetermined concentration threshold. The predetermined concentration threshold may for example be a legal limit for a drug of abuse. In such embodiments, the output of the device may be a positive result or a negative result (i.e. a concentration of the analyte above or below the threshold respectively).

In preferred embodiments, the at least one ML algorithm is configured to decrease noise present in the electrical signal for determining the presence or the absence of the analyte in the sample, the noise resulting from at least one of subject-to-subject variations in the sample, discrepancies between batches of the sensing electrode, and analog compound interference in the sample. The term “analog compound” as used herein refers to compounds that generate an electrochemical signal similar to that of the analyte. The analog compound when compared to the analyte can be a compound having a similar chemical structure, a similar three dimensional conformation, a similar oxidizable or reducible group, the same oxidizable or reducible group, a similar chemical formula or an isomer.

Tetrahydrocannabinol (THC) and cannabidiol (CBD) are isomers, which means they have the same atomic composition but different structures and hence, different bioactivities. While THC presents a phenol group, oxidizable at potentials near 0.4 V, CBD has two aromatic meta-hydroxyl groups with the same oxidizable capability, at almost the same potential as THC. Accordingly, in one example, CBD acts as an interference (i.e. analog compound) during THC electrochemical detection.

In some embodiments, the at least one ML algorithm is configured to perform at least one of a regression analysis and a classification task to determine the concentration or the presence of the analyte from the electrical signal. The classification task can be performed using one of logistic regression, soft regression, decision tree, random forest (RF), support vector machine (SVM) and an artificial neural network (ANN). The regression analysis can be performed using one of linear regression, gradient descent, polynomial regression, regularized linear model, ridge regression, lasso regression, RF and SVM. SVM and RF can be used for both the regression analysis and the classification task. In some embodiments, the at least one machine learning algorithm uses XGBoost. In some embodiments, the at least one ML algorithm comprises a plurality of different ML algorithms combined into an ensemble ML model.

Any suitable ML algorithm or combination of ML algorithms, can be selected for the present sensing methods. Non-limiting examples of ML algorithms and data processing methods (e.g. dimension reduction) encompassed by the present disclosure are described herein below.

### Feature Scaling

Feature scaling techniques include either normalization or standardization algorithms. Normalization consists of restraining values between a range of two specific numbers for example, [0,1] and [−1,1]. On the other hand, the standardization process transforms data to create a new dataset with specific mean and variance values. The most common linear feature scaling algorithms are Min-Max Scaler, Standard Scaler, Max Abs Scaler, Robust Scaler, Quantile Transformer Scaler, Power Transformer Scaler. Quantile Transformer and Power Transformer Scaler are the most used feature scaling techniques. Power Transformer Scaler and Quantile are non-linear transformers. Standard Scaler, Robust Scaler and Power Transformer are the most commonly used scaling techniques.

Linear feature scaling techniques transform a data point (xᵢ) to a new data point (xᵢ′) by using the following generalized formula:

$${x}_{t}^{′} = \frac{{x}_{i} - a}{b} \qquad Eq . 1$$

where, a and b are constant parameters, and their values and nature change for different transformers, illustrated in Table 1.

TABLE 1
Summary of common linear feature scaling parameters.

| Transformer | a | b |
|---|---|---|
| Min-Max | min(X) | max(X)-min(X) |
| Standard Scaler | $μ = \frac{{\sum }_{i = 0}^{N} {x}_{i}}{N}$ | $σ = \sqrt{\frac{{\sum }_{i = 0}^{N} {( {x}_{i} - μ )}^{2}}{N}}$ |
| Max Abs Scaler | 0 | max(\|X\|) |
| Robust Scaler | M(X) | IQR |

μ, σ, and N in Table 1 represent the mean, standard deviation, and the number of data points respectively. Interquartile range (IQR) is the range between the 1st and 3rd quartile. M(X) represents the median value of the old dataset. X is a vector consisting of all old data points and xᵢ is a single data point in the X.

The Min-Max Scaler is an appropriate feature scaling strategy for a non-Gaussian data distribution. On the other hand, The Standard Scaler is suitable for mainly normally distributed datasets and adjusts the data's mean and variance to desirable values. The Max Abs Scaler is similar in performance to the Min-Max Scaler for a dataset comprised of all positive values. The Robust Scaler removes the median value and transforms data according to the IQR; hence it is the least sensitive to marginal outliers amongst all mentioned linear transformers.

Furthermore, monotonic transformations are the principal concept of non-linear scalers like the Quantile Transformer Scaler and the Power Transformer. The former is a non-parametric transformer that uses quantile information and maps the data to a uniform or a normal distribution between 0 and 1. The latter transformer consists of a family of parametric transformations that map data to a more Gaussian-like distribution by minimizing the skewness and stabilizing variance. The Quantile Transformer Scaler performs rank transformation and usually eliminates anomalies, thus robust to outliers. Nevertheless, non-linear transformations often distort linear correlations and distances in the old datasets.

### Regression

Predicting a numerical value for one or more target outputs based on past experiences, dataset, is a typical task in ML which is called Regression. Both Regression and classification tasks require training processes with labeled output data; thus, algorithms are supervised. Linear and Polynomial Regression, Gradient Descent (GD), Regularized Regression, and Support Vector Machine (SVM) are examples of ML regression algorithms.

### Linear Regression:

Linear Regression (LR) is a simple ML regression algorithm suitable for medium-sized datasets. Eq. 2 is the generalized form of LR, where Y, β, X and ϵ represent the target (dependent variable), the matrix of regression parameters, the independent variable vector, and the error vector respectively. Finally, the ^{T} superscript is a transpose sign. Regression parameters include regression coefficients and intercept.

$$Y = {β}^{T} \cdot X + ϵ \qquad Eq . 2$$

The objective of linear regression is to calculate the best fit for the regression parameters by optimizing a cost function. A cost function is a measurement of the quality of an algorithm and can be defined based on an evaluation metric. The most common evaluation metric in ML is the mean square error (MSE) that can be defined as follows:

$$MSE ( β ) = \frac{1}{N} {\sum }_{t = 0}^{N} {( {β}^{T} {x}_{i} - {y}_{i} )}^{2} \qquad Eq . 3$$

The optimized regression parameter for minimizing the MSE can be calculated based on the Normal equation, as per Eq. 4.

$${β}_{m} = {( {X}^{T} \cdot X )}^{- 1} \cdot {X}^{T} \cdot Y \qquad Eq . 4$$

### Gradient Descent

The computational time of calculating the inverse operation (X^{T}.X)⁻¹ of a matrix with n×n dimension in the Normal Equation is approximately O(n2.4)~O(n3). Consequently, doubling the size of data or the number of independent variables can increase the computational time up to 8 times. Therefore, the gradient descent concept can be used to expedite the optimization of cost functions by adjusting parameters iteratively.

In the Gradient Descent method, initialization starts with randomly selecting values for regression parameters and then calculating the local gradient of the cost function, as per Eq. 5, and moving in the direction of descending gradient iteratively. The learning rate hyperparameter (η) is the key parameter in GD and defines the size of steps, as per Eq. 6. The smaller learning rate leads to a more time-consuming training process. However, the higher learning rate can destabilize the algorithm and result in its divergence. The GD approach may not find the desirable global optimized parameters if the cost function is not a convex function.

$${∇}_{β} MSE ( β ) = ( \begin{matrix}\frac{\partial }{\partial {β}_{o}} MSE ( β ) \\ \frac{\partial }{\partial {β}_{1}} MSE ( β ) \\ . \\ . \\ . \\ \frac{\partial }{\partial {β}_{N}} MSE ( β )\end{matrix} ) = \frac{2}{m} {X}^{T} \cdot ( X \cdot β - Y ) \qquad Eq . 5$$

$${β}^{new} = β - η {∇}_{β} MSE ( β ) \qquad Eq . 6$$

Solving the general form of Eq. 5 requires performing a derivative operation on all training data points at every step. This approach is called the Batch Gradient Descent (BGD). Alternatively, Stochastic Gradient Descent (SGD) can be utilized to find an approximate solution where the derivative operation at every step is only performed on a random data point in a training set. The SGD approach will decrease the required memory and computational time. Nonetheless, the randomness nature of the SGD leads to a close but not an exact solution. Hence, it can bounce around the optima constantly without settling down for any solution. On the other hand, randomness can be beneficial for irregular and complex cost functions by discarding local optima. Introducing an adjustable learning rate that decreases gradually during the iteration process can resolve the divergence problem in the GD. Another major category of the GD family approach is called the Mini-Batch Gradient Descent (MBGD) technique, wherein each step is a random set, and not a single point of the training set is used for gradient calculations. This may result in a closer solution to the optimal value, while the chance of trapping in local optima regions may increase compared to the SGD method.

### Polynomial Regression

Polynomial Regression is a family of linear regression models and used when the relationship between any independent variable (xᵢ) and target variable can be defined as per Eq. 7, as a polynomial equation of p^{th} degree. It should be noted that Polynomial regression also counts for variables' linear relationship, xᵢxⱼ, as well.

$$F ( {x}_{i} ) = {c}_{o} + {c}_{1} {x}_{i}^{2} + … + {c}_{p} {x}_{i}^{p} \qquad Eq . 7$$

### Regularized Linear Models

ML techniques often encounter two major challenges, underfitting and overfitting. These conditions indicate either oversimplification (underfitting) or high complexity (overfitting). The evaluation functions show poor quality for both training and testing sets in the case of underfitting that cannot be fixed by increasing the training size. On the other hand, overfitting indicates the model's high sensitivity to small variations and unwanted noise in the training set. Consequently, overfitting models often perform very well on training sets and underperform on testing sets. This problem can be resolved by increasing the size of the training data set or regularizing the model. Ridge and Lasso Regressions are widely used regularization techniques.

### Ridge Regression

Ridge Regularization (Tikhonov Regression) is used to constrain the regression model by introducing a new term into the cost function, as per Eq. 8.

$$J ( β ) = MSE ( β ) + γ {\sum }_{i = 1}^{N} {β}_{i}^{2} \qquad Eq . 8$$

Where, γ is a hyperparameter that controls the algorithm. If it is zero, then the Ridge technique will be equal to normal LR. Large γ will reduce the complexity of the model and may result in an underfitting model.

### Lasso Regression

Similarly, Least Absolute Shrinkage and Selection Operator Regression (Lasso) add an extra term to cost function, as per Eq. 9. Lasso regression removes the least important features.

$$J ( β ) = MSE ( β ) + γ {\sum }_{i = 1}^{N} \vert {β}_{i} \vert \qquad Eq . 9$$

### Support Vector Machine:

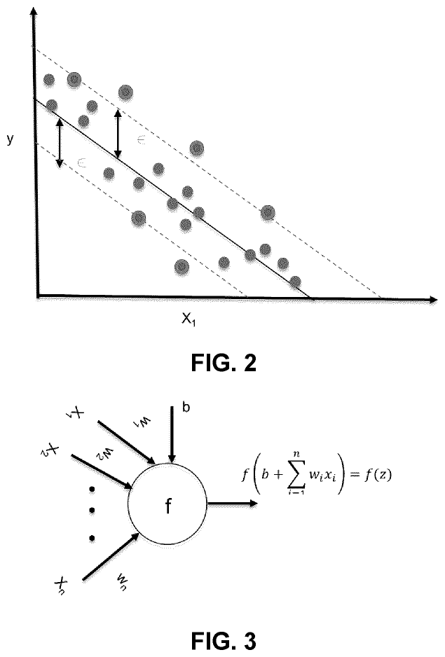

Support Vector Machine (SVM) is a ML approach particularly suitable for complex and small-medium size datasets with both classification and regression applications. Linear, Non-Linear and Kernel SVM are the three main types of SVM family for both classification and regression tasks. The main concept of all previously discussed regression techniques was to find a line that fits the training set by minimizing cost functions (least square error). At the same time, the objective of Linear SVM regression is to find an acceptable margin of error to fit the training set along an appropriate line (hyper plane). FIG. 2 demonstrates a simple univariate regression. The solid black line is the fitting line, and the two red dashed lines are at the vertical distance of v from it. The dashed lines determine the margin of error, where the error is ignored for data points in the region between two lines.

The SVR linear approach is v-insensitive since adding more instances (data points) within two dashed lines does not alter the performance of the model. The general math behind the SVR linear can be explained by the following equations. The objective is to minimize Eq. 10 while the constraint imposed by Eq. 11. is to be met. Eq. 10 is often called the primal problem.

$$\frac{1}{2} {β}^{T} \cdot β + C {\sum }_{i = 1}^{N} ‖ {ξ}_{i} ‖ \qquad Eq . 10$$

$$\vert {y}_{i} - {β}_{i} {x}_{i} \vert \leq ∈ + \vert {ξ}_{i} \vert \qquad Eq . 11$$

Where, C and ξ denote the hyperparameter and the slack variable respectively. The slack variable measures the distance of an instance (data point) from its hyperplane. Two conflicting goals are to be satisfied, finding the smallest slack value to decrease the error of the model and the highest acceptable margin of error. The hyperparameter C creates a tradeoff between these incompatible objectives.

Non-linear SVM regression models deal with datasets with a non-linear relationship between dependents and independent variables. Accordingly, in some embodiments, finding a suitable hyperplane to fit the training set requires mapping data to a higher dimension space. Kernel methods can be used to define non-linear decision boundaries (hyperplanes). Kernel functions implicitly determine the inner products of transformation functions in a high-dimensional space based on original vectors if it satisfies Mercer's condition, as per Eq. 12. According to Mercer's theorem, Kernel must be continuous and symmetrical. In other words, the Kernel function calculates the dot product of two transformation functions of two vectors in original space by circumventing the calculation of the transformation function and is only based on the vectors themselves.

$$K ( {X}_{i} , {X}_{j} ) = 〈 Φ ( {X}_{i} ) , Φ ( {X}_{j} ) 〉 \qquad Eq . 12$$

Where, K, Φ, and < > represent Kernel, the transformation function, and the dot operation product respectively. Xᵢ, and Xⱼ are the two vectors in original space. The most commonly used Kernel functions are summarized in Table 2 where γ and r represent constant coefficients.

TABLE 2
Summary of Kernel functions.

| Kernel Name | Function |
|---|---|
| Linear | ${X}_{i}^{T} \cdot {X}_{j}$ |
| Polynomial pth degree | ${( γ {X}_{i}^{T} \cdot {X}_{j} + r )}^{p}$ |
| Gaussian Radial Basis Function (RBF) | e^{(-γ\|\|x}^{ᵢ}^{-x}^{ⱼ}^{\|\|)} |
| Sigmoid | $tanh ( γ {X}_{i}^{T} \cdot {X}_{j} + r )$ |

The Gaussian RBF Kernel can transform an instance to an infinite-dimensional space since exponential functions can be extended by the famous Taylor series. γ parameter for RBF represents the impact of a single instance on the training set. The Sigmoid Kernel does not meet all the criteria of Mercer's conditions, it instead provides satisfactory results in practice.

### Classification

Classification is another major task that can be resolved by applying various supervised ML techniques, including Logistic Regression, Soft Regression, Decision Tree, Artificial Neural Network (ANN), and SVM.

### Logistic Regression

Logistic Regression is a probabilistic ML approach that can be applied for binary classification tasks. The probability of an instance to belong to a positive class can be determined via the following equation.

$$P ( X ) = \frac{1}{1 + {e}^{( - {β}^{T} \cdot X )}} \qquad Eq . 13$$

If the P(X) values from Eq. 13 are less than 0.5, the instance belongs to class 0; otherwise, it belongs to class 1. The general concept is to find an optimal value for β that creates high probabilities for positive instances and low probabilities for negative instances. Eq. 14 describes the proposed cost function for a single data point. This cost function should be applied to all instances in the training set, and the average of these cost functions should be optimized, as per Eq. 15. This cost function does not have any exact solution; nevertheless, it is a convex function. Hence, GD approaches can be utilized to find the approximate optimal parameters.

$$C ( X ) = \{ \begin{matrix}- log ( P ) & if y = 1 , \\ - log ( P ) & if y = 0\end{matrix} \qquad Eq . 14$$

$$J ( β ) = \frac{- 1}{N} {\sum }_{i = 1}^{N} [ {y}_{i} log ( {P}_{i} ) + ( 1 - {y}_{i} ) log ( 1 - {y}_{i} ) ] \qquad Eq . 15$$

### Softmax Regression

Softmax Regression is an extension of Logistic Regression for multiclassification, K classes. The basic idea is to determine the score of an instance for each class and then compute the probability of each class by applying the softmax function, as per Eq. 16. This algorithm classifies only one class at a time.

$${P}_{K} ( X ) = \frac{{e}^{( - {β}_{K}^{T} \cdot X )}}{1 + {\sum }_{j = 1}^{K} {e}^{( - {β}_{j}^{T} \cdot X )}} \qquad Eq . 16$$

### Decision Tree

Decision Tree has dual applications and can perform both regression and classification tasks. The foundation of this approach consists of a series of rules and is one of the least sensitive ML techniques to feature scaling. The process starts with dividing the entire training set into two subsets based on a threshold criterion for a single feature. The feature with the purest subsets is the selected feature. Each subset is split in the same manner until the tree reaches its predefined depth or cannot be split anymore. The cost function for this algorithm can be computed as follows:

$$J ( k , {t}_{k} ) = \frac{{N}_{l}}{N} {G}_{l} + \frac{{N}_{r}}{N} {G}_{r} \qquad Eq . 17$$

Where, k and tₖ denote the k^{th} feature and its threshold. G represents the Gini function which is a measurement of impurity. Finally, subscripts l and r symbolize left and right.

A node is pure when all its instances belong to a single class, G=0. Gini function for the i^{th} node can be determined as follows:

$${G}_{i} = 1 - {\sum }_{k = 1}^{N} {P}_{i , k}^{2} \qquad Eq . 18$$

Where, P_{i,k} is the probability of class k among training instances in the i^{th} node.

It should be noted that the decision tree algorithm determines impurity at a single level. This may not lead to an optimal solution overall; instead, it leads to a reasonably good solution. Entropy (S) is another widely used measurement for impurity, as per Eq. 19.

$${S}_{i} = - {\sum }_{k = 1}^{N} {P}_{i , k} log ( {P}_{i , k} ) \qquad Eq . 19$$

Decision Tree techniques are not abided into any predefined parameters; hence they have an adaptable structure that can very closely fit training data. This free structure increases the risk of overfitting that should be avoided by introducing regularization hyperparameters, including restricting the maximum depth of the tree, the minimum number of instances at each node before the splitting process, and the maximum number of nodes in the entire tree. A similar strategy can be used for the regression task, where the splitting criterion is determined by mean squared error at each node, as per Eq. 20.

$$J ( k , {t}_{k} ) = \frac{{N}_{l}}{N} {MSE}_{l} + \frac{{N}_{r}}{N} {MSE}_{r} \qquad Eq . 20$$

### Artificial Neural Network:

Artificial Neural Network (ANN) is a versatile and powerful ML technique with dual applications. Different families of ANN have been developed since it was introduced in the early 40s. The key element of any ANN architecture is the Perceptron (or node or neuron), as illustrated in FIG. 3. The output of a neuron without an activation function (f) will be a linear combination of weighted inputs (w, xᵢ) and an added bias (b). Hence, an activation function plays a crucial role to introduce non-linearity to the system. The most common activation functions are summarized in Table. 3. The softmax activation function is often used when a neuron has more than one output. The scaled Exponential Linear Unit (SELU) has two predefined parameters, α, and s. The weights are learnable and updated iteratively by a leaning strategy during training.

TABLE 3
Common Activation Functions.

| Activation Function | f(z) |
|---|---|
| Sigmoid (Logistic) | $\frac{1}{1 + {e}^{- z}}$ |
| Tanh | $\{ \begin{matrix}0 & if z < 0 \\ z & if z \geq 0\end{matrix}$ |
| Step | $\{ \begin{matrix}0 & if z < 0 \\ 1 & if z \geq 0\end{matrix}$ |
| Rectified Linear Unit (ReLU) | $\{ \begin{matrix}0 & if z < 0 \\ z & if z \geq 0\end{matrix}$ |
| Softmax | $\frac{{e}^{{z}_{j}}}{1 + {\sum }_{k = 1}^{K} {e}^{{z}_{k}}}$ |
| Scaled Exponential Linear Unit (SELU) | $\{ \begin{matrix}s * z & if z < 0 \\ s * α * ( 1 - {e}^{z} ) & if z \geq 0\end{matrix}$ |

Several types of neural network methods have been developed, including Multi-Layer Perceptron (MLP), Convolutional Neural Network (CNN), and Recurrent Neural Network (RNN). CNN models have been widely used in computer vision and natural learning process (NLP) applications. Predicting the output of a time-series is often performed by a RNN network.

An MLP model consists of an input layer, one or more hidden layers, and an output layer, where each layer has one or more nodes. An MPL model with two or more hidden layers is often called a Deep Learning Network (DNN). Every layer except the output layer has a bias, and all neurons are fully connected in an MPL model. The backpropagation algorithm is the main training algorithm for MLP, where the weights and bias iteratively are adjusted via a gradient descent approach to minimize the cost function. In other words, at every step, the backpropagation algorithm first predicts the output in a forward pass, then computes the error, and then calculates the error contribution from each connection through a backward pass. Finally, it applies a gradient descent approach to adjust the weights and biases.

Finding an optimized architecture for an MLP model, i.e., the number of hidden layers and neurons, is a trial-error procedure with no actual instruction. Nonetheless, some general guidelines can be used to overcome major challenges of an MLP and improve its performance. One of the main problems of the backpropagation approach is unstable gradients. When the gradients are reduced to very small values iteratively, and ultimately the gradients vanish, some weights in the network often remain unchanged. On the other hand, the weights of the layer can become very large when the gradients explode to very high values. One solution is to control the signal flow in both forward and reverse direction by equalizing the variance of output of each layer to the variance of its input and having gradients with equalized variance in both directions. The problem can be resolved by choosing the proper activation function and batch normalization. Batch normalization includes calculating the mean and standard variation of inputs and then shifting and rescaling the inputs before applying the activation function, as per Eq. 21.

$$z = ω \frac{x - μ}{\sqrt{{σ}^{2} + ε}} + δ \qquad Eq . 21$$

Where, x, μ, σ, and z represent the input, the mean, the standard deviation of inputs, and the output respectively. Additionally, ω, δ, and ε represent the scaling parameters, offset, and smoothing terms. The smoothing term is a very small positive number to tackle zero standard deviation.

Introducing a threshold for gradients can be an alternative solution to overcome the gradient exploding challenge. Stopping the training at an early stage and applying regularization techniques are the two major strategies to tackle overfitting. Dropout is a common regularization method that dedicates a probability (dropout rate) to every node. In other words, each node at each iteration has a probability to be ignored. The dropped-out nodes can reactivate in the next iteration. It should be noted that dropping out only occurs during training and not during testing.

### Ensemble ML

Combining different ML algorithms to improve the performance and seek a better predictor is called the ensemble ML approach. The performance of an ensemble model will improve with independent predictors. In other words, using different ML training algorithms on the same dataset or applying the same training algorithm on a different random subset of the main training set will increase the accuracy of an ensemble model.

### Boosting

When an ensemble algorithm aggregates various weak learning techniques and trains them sequentially, it is referred to as the boosting method. Two main boosting algorithms are adaptive boosting and gradient boosting. In adaptive boosting, the focus is on improving the underfitted instances of training. It introduces a weighting and updating strategy to instances with low accuracy as adding a new predictor to an ensemble. Therefore, the boosting technique enhances the accuracy of the ensemble model gradually, but the training process cannot be parallelized. The basic concept of the gradient boosting approach is similar to adaptive boosting, apart from the type of parameters that need to be updated. In a gradient boosting technique, the residual errors are modified by subsequently adding a new predictor. In some embodiments, the at least one machine learning algorithm described herein uses the XGBoost technique.

### Random Forest

Random Forest is a popular ensemble technique that combines a group of Decision Tree models. The model consists of various individual trees where each tree is trained on a sample of the training set with often replacement. Random Forest incorporates the hyperparameters of a Decision Tree combined with additional randomness and hyperparameter. The splitting process at each node occurs among random subsets features. This extra randomness creates a higher diversity and a better overall performance. One can increase the randomness of the model by introducing a random threshold for the features. A Random Forest model can calculate the relative importance of each feature by measuring the contribution of a feature on impurity reduction.

### Dimensionality Reduction

The training process of a highly complex dataset can include thousands and thousands of instances with dozens of features which can be extremely time-consuming. High-dimensional datasets often suffer from sparsity and overfitting. Therefore, dimensionality reduction can expedite and improve the performance of an ML technique. Additionally, it can facilitate the visualization of data to obtain a better insight of data. Principal Component Analysis (PCA), Locally Linear Embedding (LLE), Multidimensional Scaling (MDS), t-Distributed Stochastic Neighbor Embedding (t-SNE), and Linear Discriminant Analysis (LDA) are major popular techniques for dimensionality reduction.

### Principal Component Analysis

The distribution of training instances across all dimensions is not often uniform. Many features remain approximately constant, while some features show a considerable deviation that can be highly correlated. This proves the existence of a lower-dimensional subspace (hyperplane) where the entire dataset is located either on it or on a close vicinity. The basic idea is to find that specific hyperplane and then transform the original dataset to a new projected dataset on the hyperplane. Principal Component Analysis (PCA) determines the hyperplane that will not alter the maximum variance of original data; as a result, it reduces the information loss during the projection phase significantly. First, axes based on their descending amount of variance are determined. Each new axis should be orthogonal to previous axes. The unit vector of each axis is called the principal component (PC) and can be calculated based on the Singular Value Decomposition (SVD) technique. The new transformed dataset can be determined as follows:

$${X}_{new} = X \cdot {C}_{d} \qquad Eq . 21$$

Where, C_{d} denotes matrix of first d principal components.

The optimal first d component is equal to the minimum number of dimensions that maintain 95% of the original dataset's variance. Implementing the SVD technique in the original version of PCA to calculate principal component vectors requires the storage of the entire training set. Incremental, Randomized, and Kernel PCA are alternative versions of PCA that may either free memory or expedite the training process.

### Locally Linear Embedding

Locally Linear Embedding (LLE) is a nonlinear dimensionality technique for non-noisy datasets. It determines linear relationships between adjacent instances and seeks low-dimensional subspace that can maintain these correlations. LLE preserves local distances but does not maintain the distances between instances on a large scale. The first step is to identify local neighborhoods consisting of k closest instances. The second step is to redefine each instance, xᵢ, as a function of linear summation of adjacent instances in the neighborhood,

$${\sum }_{j = 1}^{k} {w}_{j} {x}_{j} .$$

wⱼ represents the weight of neighboring instance and is determined based on the optimization of

$$‖ {x}_{i} - {\sum }_{j = 1}^{k} {w}_{j} {x}_{j} ‖ .$$

In other words, in this step, the objective is to find weights while instances are fixed. Finding the optimal position of the instances in a d-dimensional subspace while weights are fixed is the final step of this technique.

### Multidimensional Scaling

Multidimensional Scaling (MDS) is another dimensionality reduction technique that finds the hyperplane while maintaining the distances between instances in the new subspace.

t-Distributed Stochastic Neighboring Embedding

The objective of the t-Distributed Stochastic Neighboring Embedding (t-SNE) approach is to create clusters of similar and dissimilar instances far away from each other.

### Linear Discriminant Analysis

Linear Discriminant Analysis (LDA) finds the most discriminative axes between different classes to define a hyper plane. As a result, after projecting the original dataset onto the new hyperplane, instances in different classes will be kept apart.

### Processing Device

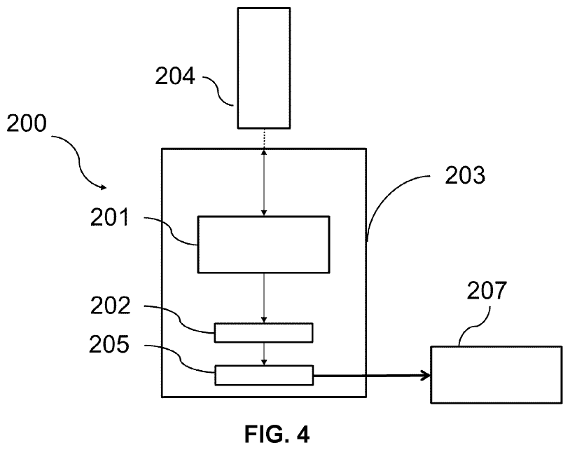

The ML algorithms are executed on the processing device. Making reference to FIG. 4, the electrochemical sensor 204 may be used with a processing device 203 to form a detection system 200, which may be handheld, portable, or fixed. For simplicity only one processing device 203 is shown but a detection system 200 may include more processing devices 203 operable by users to access remote network resources and exchange data. The processing devices 203 may be the same or different types of devices. The processing device 203 comprises at least one processor 201, a data storage device 202 (including volatile memory or non-volatile memory or other data storage elements or a combination thereof), and at least one communication interface 205. The processing device 203 components may be connected in various ways including, but not limited to, directly coupled, indirectly coupled via a network, and distributed over a wide geographic area and connected via a network (which may be referred to as “cloud computing”). It will be understood that the computing device 203 comprises all analog circuitry necessary to interface with the sensor 204.

For example, and without limitation, the processing device 203 may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

Each processor 201 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 202 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory 202 can have the at least one ML algorithm stored therein. Moreover, the memory 202 can store the training data set and may continuously update the training data set. The memory 202 may have various concentration thresholds stored therein and can store the readings performed over time.

In some embodiments, the processing device 203 is coupled to a voltage generator, and the electric potential scan is programmed on the processing device 203, for example stored in the memory 202. In such embodiments, the processing device 203 can run the electric potential scan autonomously and automatically using the processor 201.

Each communication interface 205 enables the processing device 203 to interconnect with one or more input/output devices 207, such as a keyboard, mouse, camera, touch screen microphone, display screen and speaker. For example, a display screen may display a symbol or sign that is indicative of the presence or absence of the sample analyte in the sample. In one embodiment, the display screen displays the value of the concentration of the sample analyte in the sample. In a further embodiment, the display screen may display the estimated value of the concentration of the sample analyte in the sample of the subject who provided the sample. The display screen can be a simple display in black and white or a more modern touch screen able to receive commands. A user interface may contain a button or other physical means for the user to signal to the device to begin the analysis of a sample.

In some embodiments, a network interface enables the processing device 203 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

The processing device 203 can be operable to register and authenticate users (using a login, unique identifier, and password for example) prior to providing access to applications, a local network, network resources, other networks and network security devices. The processing device 203 may serve one user or multiple users.

### Electrochemical Sensors

Examples of electrochemical sensors that benefit from the method described herein include, but are not limited to, test strips and microfluidic chips. For example, an alcohol test strip or chip, a tetrahydrocannabinol strip or chip, an opioid strip or chip, a narcotics strip or chip, a steroid strip or chip, an amphetamine strip or chip, a barbiturate strip or chip, a buprenorphine strip or chip, a methamphetamine strip or chip, a cotinine strip or chip, a PCP strip or chip, a MDMA strip or chip, a LSD strip or chip, etc. In some embodiments, the electrochemical sensor and/or the sensing electrode can be disposable.

Electrochemical Sensors with Sensing Analyte

In some embodiments, the electrochemical sensor has a sensing electrode having sensing analyte associated therewith. In this case, the sensing analyte on the sensing electrode (i.e. working electrode (WE)) improves the affinity of the electrode with the analyte present in the sample (sample analyte) through physical or chemical interactions amplifying the electrical signal obtained by electric potential scan (e.g. SWV signal).

The sensing analyte can be coated via electrodeposition of the same analyte that is expected to be detected later in the sample, although other appropriate techniques are contemplated. The choice of deposition method may vary depending on the analyte. The deposition method should limit or avoid altering the ability of the sensor analytes, once associated with the surface of the sensing electrode, to facilitate the electrochemical reaction of the sample analyte. Moreover, the deposition method should limit or avoid altering the electrochemical features (such as the conductivity) of the sensing electrode.

In some embodiments, the sensing electrode is associated or operatively coupled with a plurality of sensor analytes. As used in the context of the present disclosure, the sensing electrode is a working electrode designed to facilitate an electrochemical reaction of the sample analyte. As used in the context of the present disclosure, a sensor analyte is a chemical species or a mixture of chemical species which is/are associated (directly or indirectly) with the sensing electrode prior to the detection of the sample analyte.

The association between the sensor analyte and the sensing electrode can be caused by any physical or chemical interaction (or a combination thereof), including, but not limited to ionic interactions, covalent interactions, hydrogen interactions, van der Waals interactions, and/or electrostatic interactions. In some embodiments, the sensor analytes protrude, at least partially, from the surface of the sensing electrode. The sensor analytes can be adsorbed, at least in part, on the surface of the sensing electrode. The sensor analytes can be immobilized, at least in part, on the surface of the sensing electrode. The sensor analytes can be embedded, at least in part, in the sensing electrode.

A portion of the plurality of sensor analytes can be directly associated with the surface of the sensing electrode and/or interact directly with the surface of the sensing electrode. In some embodiments, a portion of the plurality of sensor analytes can be indirectly associated with the surface of the sensing electrode. In such embodiments, the sensor analytes can be associated with one or more sensor analytes which is directly associated with the surface of the sensing electrode. In some specific embodiments, the sensor analytes can be integrated into a dimer, an oligomer, or a polymer of one or more species of the sensor analytes in which at least one monomeric unit is directly associated with the surface of the sensing electrode.

The plurality of sensor analytes cover at least in part the surface of the sensing electrode. In an embodiment, the plurality of sensor analytes covers at least 10, 20, 30, 40, 50, 60, 70, 80, 90% or more of the surface of the sensing electrode. In an embodiment, the plurality of sensor analytes cover a majority or a totality of the sensing electrode's surface. In one embodiment, the entire surface of the sensing electrode is covered by the plurality of sensor analytes. In a specific embodiment, at least about 90, 95, 96, 97, 98, 99% or more of the surface of the sensing electrode is covered by the plurality of sensor analytes.

In some embodiments, the electrochemical sensor comprises a baseline electrode. As used in the context of the present disclosure, the baseline electrode is a working electrode designed to detect and optionally quantify the contribution of electroactive agents present in the sample which can interfere with the detection of the sample analyte. The baseline electrode corresponds to the sensing electrode prior to its association with the plurality of sensor analytes. In some embodiments, the baseline electrode is a bare working electrode.

The baseline electrode can include any suitable conductive material and can be made of the same material as the sensing electrode (without the sensor analytes). In one embodiment, the baseline electrode comprises a carbon-based material, a nanomaterial, a metal-based material, or a combination thereof. In one embodiment, the baseline electrode comprises carbon, gold, platinum, palladium, ruthenium, rhodium, or a combination thereof. In a further embodiment, the baseline electrode may be a screen-printed electrode (SPE). The baseline electrode may be of any shape or size. Known SPE include, but are not limited to, a Zensor electrode, a Dropsens electrode, a Zimmer Peacock electrode, Flex Medical Electrode or a Kanichi electrode. In one embodiment, the baseline electrode is a Zensor carbon-based electrode.

In some embodiments, the electrochemical sensor includes one or more reference electrodes. In an embodiment, each working electrode (i.e. the sensing electrode and the baseline electrode) can be associated with one or more reference electrodes. In another embodiment, two or more working electrodes can be associated with the same reference electrode. The reference electrode is an electrode with a stable and well-defined electrochemical potential against which the potential of other electrodes like the sensing electrode or baseline electrode can be controlled and measured. When the reference electrode is in use, it is intended to be covered by the sample. In one embodiment, the reference electrode comprises or consists of silver. When the reference electrode is screen printed, it can be prepared with Ag/AgCl ink or Ag ink.

In some embodiments, the sensor includes one or more counter electrodes. In an embodiment, each working electrode can be associated with one counter electrode. In another embodiment, two or more working electrodes can be associated with the same counter electrode. The counter electrode completes the circuit of a three-electrode cell, as it allows the passage of current. After the sample is placed on a sample receiving region, a potential is applied between the sensing electrode and the reference electrode, and the current induced is measured. At the same time, a potential between the counter electrode and the reference electrode is induced which will generate the same amount of current (reverse current). Therefore the sensing electrode, baseline electrode, reference electrode, and counter electrode are all intended to be in fluid communication with the sample. The counter electrode can be made of the same materials as the sensing electrode and/or the baseline electrode and/or the reference electrode. In one example, the counter electrode comprises or consists of carbon ink or platinum.

### Example

### Materials and Methods

In this Example, healthy human saliva samples were obtained from human donors. TE100 Screen-printed electrodes (SPE) with carbon-based working (3 mm/0.071 cm²) and counter electrodes, and silver reference were purchased from Zensor R&D. Henceforth; the SPEs will be referred to as Zensor. (−)-trans-Δ⁹-tetrahydrocannabinol (THC) and cannabidiol (CBD) standard solutions in methanol (1 mg/mL) were purchased from Cerilliant—Sigma-Aldrich. Enzyme-linked immunosorbent assay (ELISA) THC Oral Fluid Kit Product No. 120519 was purchased from Neogen Corporation. The electrochemical measurements were performed with a mono-potentiostat PalmSens4 and an EmStatMUX8-R2 Potentiostat with an integrated multiplexer for eight channels driven by the PSTrace 5-Palm-Sens software.

Manufacturing of THC Modified Zensor Electrodes (m-Z-THC)

First, the Zensor electrodes were thoroughly washed with Milli-Q water and dried with hot airflow. Next, stock solutions of THC were prepared by adding THC (1 mg/mL in methanol) in a mix of solvent methanol/water (3:1 ratio in volume). Immediately after, 1 μL of the previous stocks were dispensed on the working area of the Zensor electrodes to obtain different amounts of 100, 130, and 150 ng of the sensing analyte. Further, the electrodes were dried at room temperature (RT) airflow for 30 seconds and warm airflow for 5 seconds. Afterward, the electrodes with the analyte deposited were submitted in phosphate buffered saline (PBS) 0.01 M to perform an electrochemical treatment by using square wave voltammetry (SWV) with the following conditions: precondition potential of 0.05 V for 30 s, equilibration time of 3 s, voltammetric potential scan from 0 to 0.8 V with a frequency of 15 Hz, an amplitude of 25 mV, and a step potential of 5 mV to obtain modified Zensor (m-Z-THC) electrodes. After each recording, the m-Z-THC electrodes were thoroughly washed with Milli-Q water and stored at 4° C. degree in N₂ riched package until they were ready to be used.

### Collection and Treatment of Saliva Samples

Saliva from human donors was spat inside 15-50 mL tubes, adequately sealed with parafilm, and labeled with the donator's name and date. The saliva samples were frozen at −20° C. for long-term storage or cooled at 4° C. be tested within a period of 24 hours.

The saliva samples were dispensed in 1.5 mL Eppendorf vials. Next, the samples were spiked with an adequate amount of THC in 0.01 mL of methanol to obtain final concentrations of 0, 2, and 5 ng/mL of THC. After that, an absorbent material swab was introduced inside the vial to collect the sample.

The swabs with the absorbed saliva were introduced within a device which contained an appropriate filter and then squeezed with a plunger. The filtered saliva samples were collected in another vial to be prepared for the testing. Table 4 summarizes different swabs providers and tested filters.

TABLE 4
Examples of filters, swabs, and collectors used
to collect and filtrate the saliva samples.

| Filters-diameter-pore size | Swabs/collectors |
|---|---|
| PTFE-25 mm-0.2 μm | PureSal/Filtration(Swab + squeeze) |
| PES-25 mm-0.2 μm | NeoSal (Swab + buffer) 1:4 |
| PVDF-25 mm-0.2 μm | SalivaBio swab (Swab + squeeze) |
| Nylon-25 mm-0.2 μm | SalivaBio swab + Pure Sal filter |
| Nylon-25 mm-0.45 μm | POREX OFCD-100 (No filter) |
| Nylon-13 mm 0.45 um* | POREX OFCD-201-SRF (with filter) |
| wwPTFE NanoSEP-0.2 μm* | POREX OFCD-100 + glass wool |
| wwPTFE NanoSEP-0.45 μm* | POREX OFCD-100 swab + glass wool |
| wwPTFE-13 mm-0.45 μm* | N/A |
| wwPTFE-13 mm-0.2 μm* | N/A |
| wwPTFE-25 mm-0.2 um* | N/A |
| Glass wool (Pyrex 3950) | |

*= Pall company

### THC Sensor Performance

The collected THC samples were prepared by adding methanol. Then, 100 μL of the samples were added on electrodes and immediately after, SWV was recorded with the following conditions: precondition potential of 0.05 V for 30 s, equilibration time of 3 s, voltammetric potential scan from 0 to 0.8 V with a frequency of 15 Hz, an amplitude of 25 mV, and step potential of 5 mV. Different situations such as using or not using pre-filtration, testing different batches of electrodes, reading with mono-potentiostat and multichannel-potentiostat types of equipment, among others, were evaluated for from the samples of the different saliva donors. In a traditional analysis, the concentration of the analyte is determined with the values of the currents, by subtracting the intensity of the current peaks for the samples recovered with m-Z-THC (sensing electrode) minus the current signal obtained with the baseline electrode pristine (pristine Zensor, p-Z). Table 5 summarizes all the experimental conditions during the sensor data collection. It was also possible to eliminate methanol in the production process. The SVM method for the classification of three concentrations of THC, i.e. 0 ng, 2 ng and 5 ng, without methanol. The training and testing accuracies were 100% and 71%, respectively.

TABLE 5
Experimental conditions of the electrochemical
sensor for THC detection 0, 2, and 5 ng/mL.

| | | | | | | Amount of |
|---|---|---|---|---|---|---|
| | Pre- | THCi | | Collection | | electrodes |
| Batch | treatment | (ng) | Equipment | Collection/Filtration | Samples | P-Z/m-Z-THC |
| 1 | washing | 100 | P | Porex/GW | 1SS + 5RS | 2/5-6 |
| | | 130 | | | | |
| | | 150 | | | | |
| 2 | cleaning | 130 | P | Porex/GW | 4 RS | 2/5-6 |
| 3 | washing | 130 | P | Porex/GW | 4 RS | 2/5-6 |
| 3 | washing | 130 | P and MP | Swab Porex/GW | 6 RS | 2/5-6 |
| 2 | washing | 130 | P | Swab Porex/GW | 4 RS | 1/3 |
| 4 | washing | 130 | P and MP | Swab Porex/GW | 6 RS | 1/3 |
| 4 | washing | 130 | P | Swab Porex/GW | 20 RS | 1/3 |

P (mono-potentiostat PalmSens4), MP (multichannel EmStatMUX8-R2 Potentiostat), Porex OFCD-100 (commercial collector kit), Swab Porex (using just the swab of the collector), GW (glass wool, Pyrex 9350), SS (simulated saliva), RS (real saliva).

### THC and Cannabidiol (CBD) Electrochemical Sensor Fabrication.

In the THC-based sensor, the working electrode was modified with THC molecules (as described above for m-Z-THC) and in the case of a CBD-based sensor, the working electrode was modified with CBD molecules (m-Z-CBD).

The THC and CBD-based sensors were prepared following the same approach methodology detailed above. Briefly, before THC or CBD deposition, the Zensor electrodes were washed with Milli-Q water and dried with hot airflow. Following drying, stock solutions of THC or CBD (50-150 μg/mL) were prepared by adding THC or CBD solution (1 mg/mL in methanol) into a mix of methanol/water solvent. Subsequently, 1 μL of the stock solution was dropped onto the WE surface of the Zensor electrodes and left to dry at room temperature airflow for 30 seconds and warm airflow for 5 seconds. The obtained modified electrodes have an initial THC (m-Z-THC) or CBD (m-Z-CBD) deposition of 130 and 100 ng, respectively. Subsequently, the modified electrodes were submitted to electrochemical treatment using square wave voltammetry (SWV) with 0.01 M PBS solution. The following conditions were employed to record the electrochemical measurement: precondition potential of 0.05 V for 0 s, equilibration time of 3 s, voltammetric potential scan from 0 to 0.8 V with a frequency of 15 Hz, an amplitude of 25 mV, and step potential of 5 mV. In this case, the intensity of the current was proportional to the amount of THC-deposited on the WE. This value was registered for each electrode and henceforth will be referred to as ITHCi for THC or ICBDi for CBD. After each recording, the modified Zensor electrodes (m-Z THC and m-Z CBD) were thoroughly washed with Milli-Q water and stored at 4° C. degrees under nitrogen.

### Detection of Drugs in Human Saliva Samples Using THC and CBD Based-Sensors

Fresh human saliva was provided by healthy human donors, which was collected by spitting it into a sterilized container. The samples were kept at 4° C. in a refrigerator when not in use. The saliva sample from each donor was vortexed for 5 min each before its use. THC or CBD was spiked in low concentration levels in methanol (0, 2, and 5 ng/mL) into saliva samples. The saliva samples collection and preparation were done following the same protocol above. Then, 50 μL of the THC-spiked samples were dropped onto m-Z-THC electrodes followed by SWV interrogation under a precondition potential of 0.05 V for 30 s, equilibration time of 3 s, voltammetric potential scan from 0 to 0.8 V with a frequency of 15 Hz, an amplitude of 25 mV, and step potential of 5 mV. After that, another 50 μL from the same THC spiked sample was added to a pristine Zensor and analyzed under the same electrochemical conditions employed for the m-Z-THC measurement. The same procedure was carried out for m-Z-CBD and CBD-spiked samples. The subtracting of current methodology was employed as explained above. Briefly, the intensity of pristine Zensor (I p-Z) was subtracted from the intensity of m-Zensor (I m-Z-THC or I m-Z CBD) to minimize subject-to-subject variation (i.e., saliva-to-saliva variation in this Example).

### Interference Studies

Since the CBD molecule presents the same chemical formula as THC but with atoms arranged differently, it is desirable to evaluate the applicability of both sensors in the analysis of samples with both molecules. Therefore, the performance of the m-Z-THC sensor to detect THC in the presence of CBD was evaluated, and the ability of CBD detection by the m-Z-CBD sensor in the presence of THC was also studied. The response for both sensors was recorded before and after adding increasing amounts of each compound to a solution containing 0, 2, and 5 ng/mL of THC or CBD using SWV with the same operating parameters as described above. Several experiments were performed to determine the effect of CBD or THC on the electrochemical performance of the modified electrodes (m-Z-THC and m-Z-CBD) during the THC or CBD detection (Table 6 below).

TABLE 6
Interference experiments detail.

| | THC based- Sensor | | CBD based-Sensor |
|---|---|---|---|
| | (m-Z-THC) | | (m-Z-CBD) |
| | Total electrodes | | Total electrodes |
| | P-Z/m-Z | | P-Z/m-Z |
| Experiments | (1 Saliva) | Experiments | (1 Saliva) |
| [THC] = 0 ng/mL | 8 (1/7m-Z-THC) | [CBD] = 0 ng/mL | 8 (1/7m-Z-CBD) |
| [CBD] = 0 ng/mL | | [THC] = 0 ng/mL | |
| [THC] = 0 ng/mL | 4 (1/3m-Z-CBD) | [CBD] = 0 ng/mL | 4 (1/3 m-Z-CBD) |
| [CBD] = 10 ng/mL | | [THC] = 10 ng/mL | |
| [THC] = 0 ng/mL | 4 | [CBD] = 0 ng/mL | 4 |
| [CBD] = 50 ng/mL | | [THC] = 50 ng/mL | |
| [THC] = 2 ng/mL | 8 | [CBD] = 2 ng/mL | 8 |
| [CBD] = 0 ng/mL | | [THC] = 0 ng/mL | |
| [THC] = 2 ng/mL | 4 | [CBD] = 2 ng/mL | 4 |
| [CBD] = 10 ng/mL | | [THC] = 10 ng/mL | |
| [THC] = 2 ng/mL | 4 | [CBD] = 2 ng/mL | 4 |
| [CBD] = 50 ng/mL | | [THC] = 50 ng/mL | |
| [THC] = 5 ng/mL | 8 | [CBD] = 5 ng/mL | 8 |
| [CBD] = 0 ng/mL | | [THC] = 0 ng/mL | |
| [THC] = 5 ng/mL | 4 | [CBD] = 5 ng/mL | 4 |
| [CBD] = 10 ng/mL | | [THC] = 10 ng/mL | |
| [THC] = 5 ng/mL | 4 | [CBD] = 5 ng/mL | 4 |
| [CBD] = 50 ng/mL | | [THC] = 50 ng/mL | |
| Total of electrodes | 48 | N/A | 48 |
| (1 Saliva) | | | |

| | |
|---|---|
| Total of electrodes | 576 |
| (6 Salivas) | |

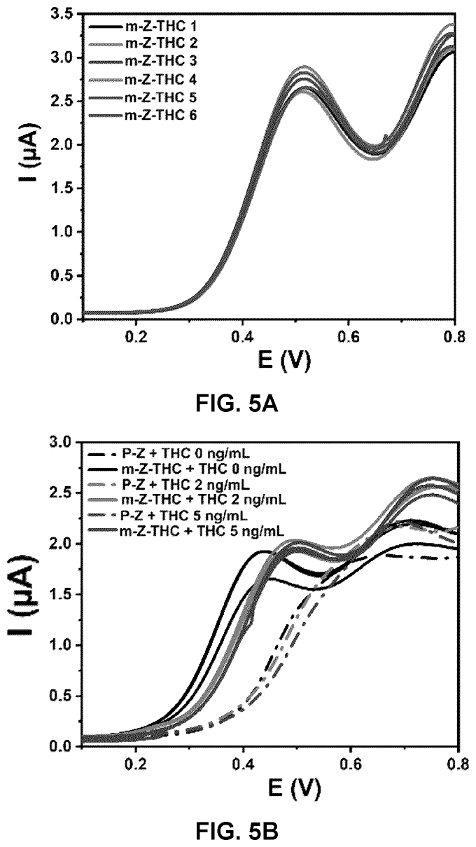

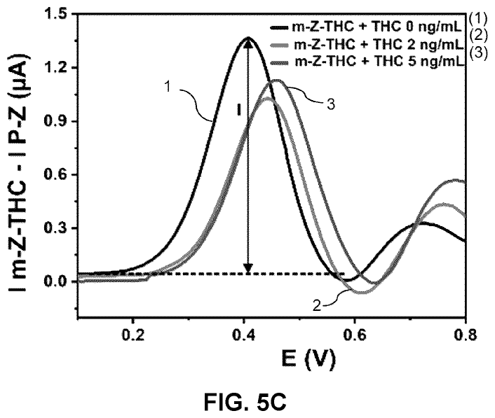

FIG. 5A shows SWV signals during the THC deposition of different modified electrodes (m-Zensor). FIG. 5B shows raw data of 3 m-Zensor and one pristine (P-Z) per THC concentration 0, 2, and 5 ng/mL. FIG. 5C is an example of the subtraction of the signals for the samples (THC 0, 2, and 5 ng/mL) recovered with m-Z-THC minus the signal obtained with pristine Zensor. In a traditional analysis, the intensity of I regarding the baseline is correlated with the THC concentration in the sample. The biomolecule-free electrochemical approach detected THC in PBS (1.1 ng/mL), simulated saliva (1.6 ng/mL), and real saliva (1.6 ng/mL). Many experimental conditions were studied during the manufacturing, sample preparation, and sensor performance. The validation and recovery of THC detection in real saliva were tested with suitable results for four real saliva samples and THC concentrations of 0, 2, and 5 ng/mL. Finally, the next step to evaluate the proposed electrochemical approach was tested with various individuals' saliva samples and hence, different saliva properties and compositions.

Saliva viscosity and natural composition can disrupt the electrode performance controlled by adsorption processes. Thus, electroactive molecules, proteins such as mucin, or supernatant solids should be eliminated to decrease the variability amongst the results. For the purpose of the present example, it is desirable for the THC concentration to be invariant in all processes. For this reason, an optimization of the saliva collection and filtration was tested. Table 7 summarizes the values of THC recoveries in saliva samples after being collected or filtered and quantified by ELISA THC Oral Fluid Kit Product from Neogen Corporation.

TABLE 7
Results of the THC recoveries in saliva samples with THC 5 ng/mL after collection or filtration.

| Filters | | Collectors | |
|---|---|---|---|
| | THC Recovery | | THC Recovery |
| Type | (%) | Type | (%) |
| PTFE-25 mm-0.2 μm | 0 | PureSal/Filtration(Swab + squeeze) | 7 (±13) |
| PES-25 mm-0.2 μm | 0 | NeoSal (Swab + buffer) 1:4 | 16 (±5) |
| PVDF-25 mm-0.2 μm | 0 | SalivaBio swab (Swab + squeeze) | 84 (±24) |
| Nylon-25 mm-0.2 μm | 0 | SalivaBio swab + Pure Sal filter | 72 (±20) |
| Nylon-25 mm-0.45 μm | 0 | POREX OFCD-100 (No filter) | 94 (±3) |
| Nylon-13 mm-0.45 um* | 7 (±7) | POREX OFCD-201-SRF (with filter) | 64 (±3) |
| wwPTFE NanoSEP-0.2 μm* | 9 (±16) | POREX OFCD-100 + glass wool | 75 (±5) |
| wwPTFE NanoSEP-0.45 μm* | 0 | POREX OFCD-100 swab + glass wool | 75 (±5) |
| wwPTFE-13 mm-0.45 μm* | 0 | Centrifuged | 91 (±19) |
| wwPTFE-13 mm-0.2 μm* | 76 (±20) | N/A | N/A |
| wwPTFE-25 mm-0.2 um* | 64 (±7) | N/A | N/A |
| Glass wool (Pyrex 3950) | 76 (±5) | N/A | N/A |

*Pall company

The wwPTFE Filter 0.2 μm and POREX OFCD-201-SRF (with filter) helped clean the saliva but presented low volume recoveries. The SalivaBio and POREX OFCD-100 was unsuccessful in cleaning the saliva; providing almost neat saliva. Contrastingly, the PureSal was successful in cleaning the saliva however, resulted in losing the THC in the swab. The SalivaBio Swab+PureSal Filter resulted in interacting with the samples leading to electrochemical interferences and a strong signal around 0.4 V, like THC. Lastly, the POREX OFCD-100+Glass wool deemed successful in cleaning the saliva but was difficult to squeeze and hence, resulted in compromising the volume recovery.

It was found that the preferred system was using the swab of the collector OFCD-100 and after filtration with glass wool (Pyrex 9350). In this case, such a combination cleans the saliva samples, presents suitable volume recovery, and has no electrochemical interference. From this point, all experiments were performed by using this strategy.

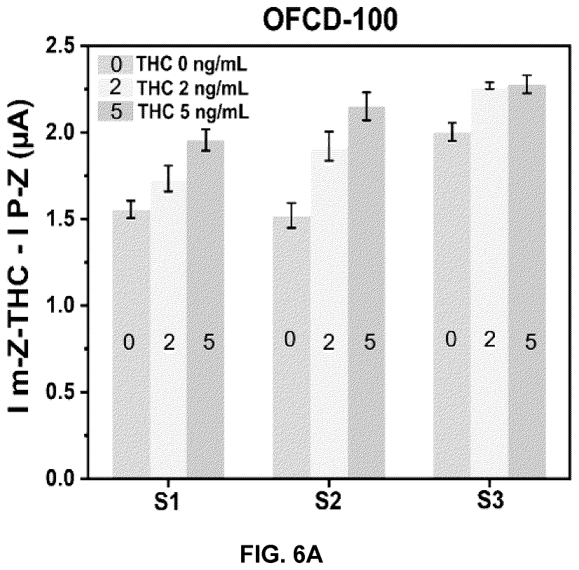

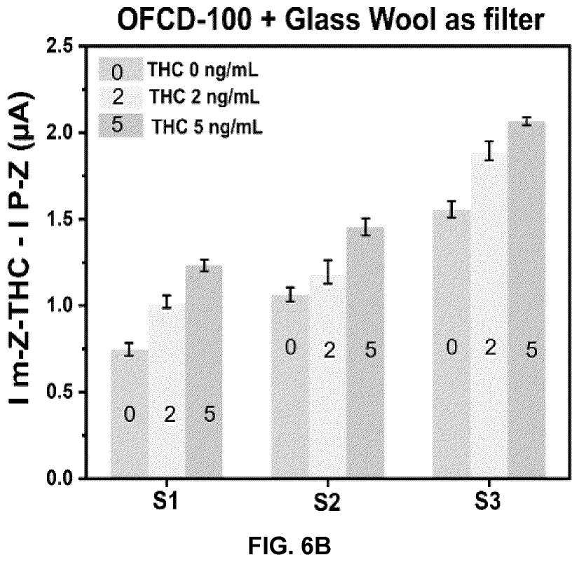

FIGS. 6A and 6B show the difference in the electrochemical performance of the sensor with THC-saliva samples 0, 2, and 5 ng/mL collected with the swab OFCD-100, filtered and unfiltered with glass wool. After the additional filtration, the intensities corresponding to each concentration depicted better differentiation and fewer interferences contributions. However, even after cleaning the saliva, there were inconsistencies in the results when comparing the current values of the same concentration but in different individual samples. For example, in FIG. 6B, the saliva sample 1 (S1) THC 2 ng/mL presented the same response as saliva sample 2 (S2) THC 0 ng/mL and so on, making the sensor inoperable.

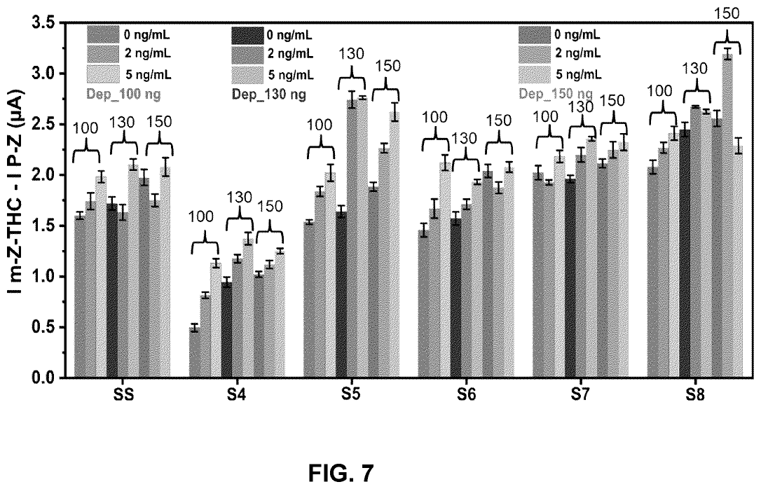

Afterward, different amounts of THC initially deposited on the m-Zensor were evaluated, looking for a suitable consistency between the intensities of the samples regarding the THC concentration. FIG. 7 summarizes the values of THC 0, 2, and 5 ng/mL in different saliva samples, including one synthetic saliva (SS) and five saliva samples from donors (S4-S8) and THC depositions of 100, 130, and 150 ng in each sample.

The results show inconsistencies and no clear tendency while testing the different THC concentrations. For the 100 ng deposition, there were differentiations between the THC concentrations for all samples (except S7, which presents a high zero value). However, for 130 and 150 ng, there was no existent correlation between the intensities and the THC concentrations in the majority of the samples.

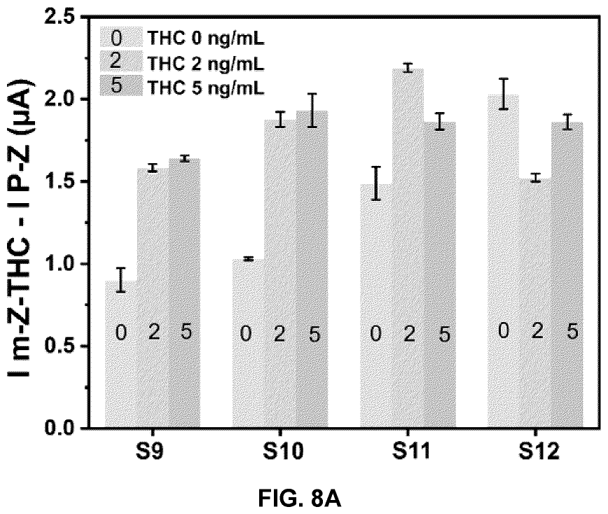

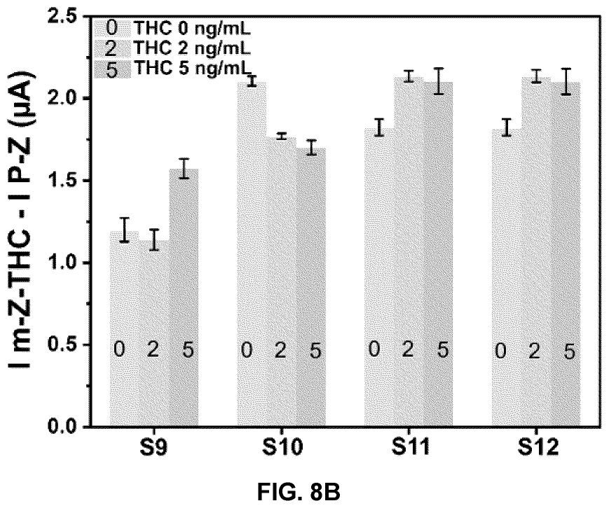

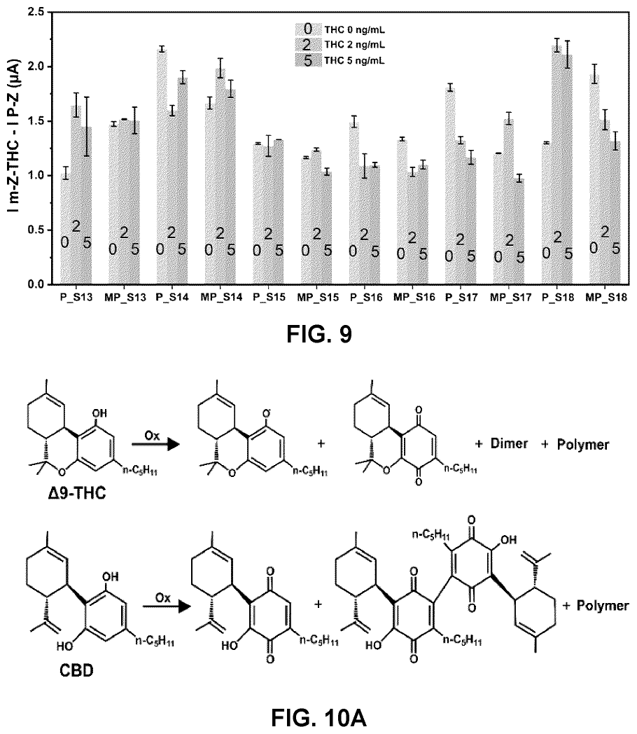

In addition to the drawback of saliva to saliva variations, despite using collection/filtration and different THC deposit amounts, discrepancies were observed while working with different batches of the commercial electrodes Zensor (FIGS. 8A-8B). Moreover, reading from different potentiostats (mono-potentiostat and multichannel potentiostat), also lead to remarkably different values due to variations during the handling and samples exposure times (FIG. 9).

### CBD as Interference of the THC Electrochemical Sensor

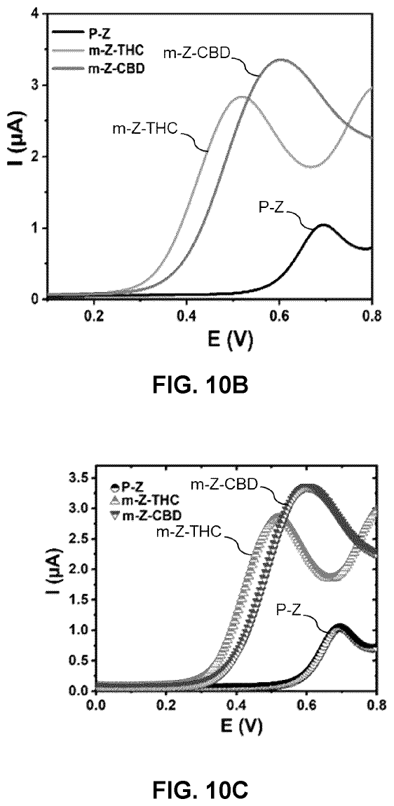

Both sensors, m-Z-THC, and m-Z-CBD, manufactured as described in the present Example were created based on the oxidation of one hydroxyl group in the THC molecule and two hydroxyl groups in CBD molecule under an applied potential to form C═O moieties followed by the formation of quinones, adducts, or more complex structures (FIG. 10A). The presence of oxidized THC or CBD on the final working electrode in the sensors facilitates further oxidation of other THC or CBD molecules present in the sample due to possible peer interactions between the analyte in the sample (THC or CBD) and the modified working electrodes in the proposed sensors (m-Z-THC and m-Z-CBD). To determine the electrochemical behavior of bare Zensor and modified Zensor electrodes, the SWV was performed in PBS solution. After modifying pristine Zensor electrodes, an oxidation peak between 0.4 and 0.7 V appears in m-Z-THC and m-Z-CBD sensors (FIG. 10B). The peak related to m-Z-CBD appeared at higher intensity and potential than the peak in the m-Z-THC sensor, which may be because, in the CBD molecule, two electrons are involved in the electrochemical oxidation, whereas in the THC molecule, only one electron is involved.

Both sensors, the m-Z-THC and the m-Z-CBD developed herein, were designed based on the oxidation of the hydroxyl group present in THC and CBD molecules under an applied potential to form C═O moieties followed by the formation of quinones, adducts, or more complex structures (FIG. 10A). The presence of THC or CBD species in the sensors (m-Z-THC and m-Z-CBD) enhanced further physical and chemical interactions of the working electrodes with the THC or CBD molecules present in the sample, hence the oxidation process. To determine the electrochemical behavior of bare Zensor and modified Zensor electrodes, the SWV was performed in PBS solution. After modifying the pristine Zensor electrodes, an oxidation peak between 0.4 and 0.7 V appeared in the m-Z-THC and the m-Z-CBD sensors (FIG. 10C). The peak related to m-Z-CBD appeared at slightly higher potentials than THC.

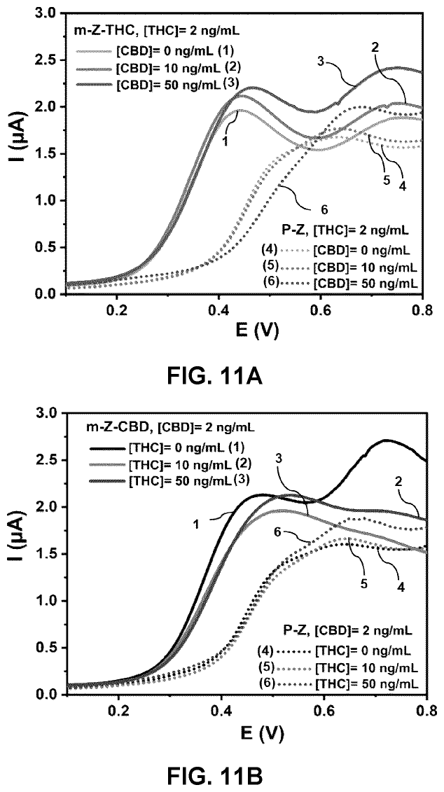

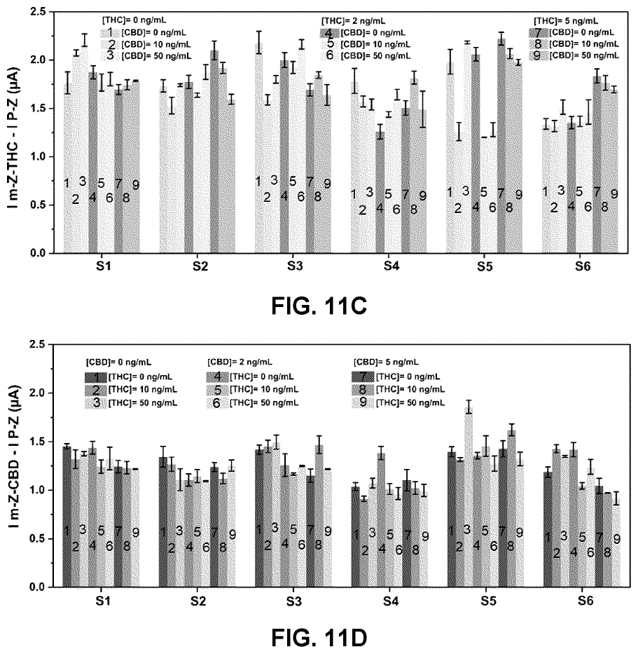

The selectivity of designed sensors was evaluated through the effect of CBD (THC) molecules in the determination of THC (CBD). The impact of CBD on THC detection was studied employing the m-Z-THC sensor and the effect of THC in the m-Z-CBD sensor when CBD is detected. FIG. 11A illustrates an example of the raw data obtained in detecting THC (2 ng/mL) in the presence of different amounts of CBD (0, 10, and 50 ng/mL) using the m-Z-THC sensor. After the analyses, three well-defined peaks appeared between 0.4 and 0.6 V with an intensity higher than 2 μA. A shift to higher potential values was observed when 50 ng/mL of CBD was employed as an interference. As shown in FIG. 11B, the peak for CBD appeared at higher potential values than THC signals when the electrochemical oxidation of these molecules was carried out; therefore, the presence of CBD in the sample can provoke the change observed in the THC signal potential. Similar signals were observed when the effect of THC presence in CBD detection using m-Z-CBD was studied (FIG. 11B). In this case, the three peaks evidenced after analyses appeared between 0.4-0.6 V and the intensity of the signals around 2 μA. However, the peaks when THC was employed as interference were broader, and a shift in the potential was observed for both interfering concentrations (10 and 50 ng/mL). This shift was more significant when 50 ng/mL of THC was employed.

Six samples from different healthy human donors were used in the experiments. FIG. 11C shows the results of THC detection in the presence of CBD using the m-Z-THC sensor. The intensity of the signals remained between 2-2.5 μA after subtraction. However, as can be seen, the signals could not be differentiated when the sample was analyzed with a different amount of the target analyte and interfering molecule (FIG. 11C). Similar behavior was observed when the m-Z-CBD sensor was employed for CBD detection under the presence of THC concentrations. In this case, the intensity of the signals after subtraction was between 1-2 μA lower than the signal obtained when the m-Z-THC sensor was employed for THC detection. The similarities in the chemical structures of THC and CBD and the saliva to saliva variation (person to person variation) can be the two principal factors that lead to the inability to differentiate between the signals obtained from the different experiments carried out. However, the influence of these two factors on the final results can be corrected using ML as will be demonstrated below.

### ML Algorithms

Random Forest (RF), Support Vector Machine (SVM), and Artificial Neural Network (ANN), are three versatile ML algorithms that were used for the classification task. These methods were applied to the entire or only a portion of the dataset to measure the impact of experimental features and the limits of individual ML techniques. The whole dataset has 1124 instances and consists of signals corresponding to all variations of experimental features (Table 5), including samples with glass wood collectors and without, different batches of electrodes, different THCi concentrations, and the type of saliva, as per Table 8.

TABLE 8
Description of different datasets used
for training for m-Z-THC sensors

| Dataset | Size | Comments |
|---|---|---|
| Df1 | 1124 | The entire dataset |
| Df2 | 773 | Instances with collectors only |
| Df3 | 701 | Instances with collectors and real saliva |
| Df4 | 469 | Instances with collectors, real saliva, |
| | | and THCi 130 ng. |
| Df5 | 122 | Instances with collectors, real saliva, |
| | | THCi 130 ng, and batch 4 electrodes. |

Moreover, proper selection of signal features can play a critical role in the success of a ML model. As a result, ML techniques were trained with only statistical features of signals, including the maximum, minimum, distance between the maximum and the minimum, mean, variance, skewness, and kurtosis or the entire signal (FIG. 5). Different dimensionality reduction techniques were used on the whole signal. Furthermore, the effect of feature scaling on ML techniques was studied. The training sets for all techniques were split into training and testing. The results for instances with only statistical features on different datasets are summarized in Table 9. The results indicate that RE performed considerably better than SVM and ANN when trained with only statistical features. Nevertheless, it seemed to have suffered from overfitting since the differences between the accuracies of training and testing sets were significant.

TABLE 9
Accuracy of ML techniques on different datasets trained
with signal statistical features for m-Z-THC sensors

| Model | Dataset | Features | Preprocessing | Train | Test |
|---|---|---|---|---|---|
| RF | Df1 | Statistical | — | 95.55 | 69.33 |
| RF | Df2 | Statistical | — | 100 | 69.68 |
| RF | Df3 | Statistical | — | 100 | 60.99 |
| RF | DF4 | Statistical | — | 100 | 62.76 |
| RF | DF5 | Statistical | — | 100 | 60 |
| SVM | Df1 | Statistical | — | 55.28 | 52.88 |
| SVM | Df1 | Statistical | RobustScaler | 54.51 | 51.11 |
| SVM | Df2 | Statistical | — | 53.07 | 52.26 |
| SVM | Df2 | Statistical | StandardScaler | 55.02 | 54.84 |
| SVM | Df3 | Statistical | — | 49.46 | 48.23 |
| SVM | Df3 | Statistical | RobustScaler | 54.82 | 51.06 |
| SVM | Df4 | Statistical | — | 52.26 | 44.68 |
| SVM | Df4 | Statistical | StandardScaler | 55.46 | 45.74 |
| SVM | Df5 | Statistical | — | 61.86 | 48 |
| SVM | DF5 | Statistical | PowerTransformer | 59.79 | 56 |
| ANN | Df1 | Statistical | — | 33.59 | 28.88 |
| ANN | Df1 | Statistical | RobustScaler | 48.83 | 44 |
| ANN | Df2 | Statistical | — | 35.43 | 31.67 |
| ANN | Df2 | Statistical | PowerTransformer | 37.21 | 34.38 |
| ANN | Df3 | Statistical | — | 35 | 29.78 |
| ANN | Df3 | Statistical | StandardScaler | 33.92 | 34.75 |
| ANN | Df4 | Statistical | — | 33.86 | 41.50 |
| ANN | Df4 | Statistical | StandardScaler | 32.8 | 38.30 |
| ANN | Df5 | Statistical | — | 34.93 | 34.04 |
| ANN | DF5 | Statistical | PowerTransformer | 35.52 | 41.48 |

Table 10 summarizes applying RE, SVM, and ANN techniques on the entire signals. The results demonstrated significant improvements in the accuracy of ML techniques trained over the entire signal features with dimensionality reduction and preprocessing. Moreover, all ML techniques perform remarkably better on a portion of datasets with the least experimental variation, i.e., df5. The variations in signal shapes for df5 datasets represent mainly saliva variation.

TABLE 10
Accuracy of ML techniques on different datasets trained
with the entire signal for m-Z-THC sensors.

| | | | | Dimensionality | | |
|---|---|---|---|---|---|---|
| Model | Dataset | Features | Preprocessing | Reduction | Train | Test |
| RF | Df1 | All | — | — | 95.55 | 75.11 |
| RF | Df1 | All | — | PCA | 95.55 | 86.67 |
| RF | Df2 | All | — | — | 100 | 76.13 |
| RF | Df2 | All | — | PCA | 100 | 91.61 |
| RF | Df3 | All | — | — | 100 | 69.50 |
| RF | Df3 | All | — | PCA | 100 | 90.78 |
| RF | Df4 | All | — | — | 100 | 58.51 |
| RF | Df4 | All | — | PCA | 100 | 85.11 |
| RF | Df5 | All | — | — | 100 | 76 |
| RF | Df5 | All | — | PCA | 100 | 92 |
| SVM | Df1 | All | — | LDA | 77.41 | 62.67 |
| SVM | Df1 | All | — | PCA | 75.64 | 65.33 |
| SVM | Df1 | All | StandardScaler | PCA | 90.01 | 81.33 |
| SVM | Df1 | All | RobustScaler | PCA | 89.54 | 78.67 |
| SVM | Df1 | All | PowerTransformer | PCA | 90.32 | 80.44 |
| SVM | Df2 | All | PowerTransformer | — | 60.68 | 58.71 |
| SVM | Df2 | All | — | LDA | 82.68 | 60 |
| SVM | Df2 | All | — | PCA | 77.83 | 69.03 |
| SVM | Df2 | All | PowerTransformer | PCA | 97.25 | 83.88 |
| SVM | Df3 | All | PowerTransformer | — | 63.04 | 52.48 |
| SVM | Df3 | All | — | LDA | 85.35 | 60.99 |
| SVM | Df3 | All | PowerTransformer | LLE | 86.25 | 69.50 |
| SVM | Df3 | All | StandardScaler | PCA | 94.64 | 77.31 |
| SVM | Df3 | All | PowerTransformer | PCA | 97.32 | 79.43 |
| SVM | Df4 | All | — | LDA | 95.73 | 63.83 |
| SVM | Df4 | All | PowerTransformer | LLE | 88.80 | 60.63 |
| SVM | Df4 | All | — | PCA | 80 | 57.45 |
| SVM | Df4 | All | StandardScaler | PCA | 98.4 | 65.96 |
| SVM | Df5 | All | StandardScaler | — | 78.35 | 56 |
| SVM | Df5 | All | — | LDA | 100 | 64 |
| SVM | Df5 | All | PowerTransformer | LLE | 98.89 | 76 |
| SVM | Df5 | All | — | PCA | 83.51 | 76 |
| SVM | Df5 | All | PowerTransformer | PCA | 98.96 | 84 |
| ANN | DF1 | All | — | — | 58.28 | 51.55 |
| ANN | Df1 | All | PowerTransformer | — | 76.08 | 59.56 |
| ANN | Df2 | All | — | — | 57.92 | 45.16 |
| ANN | Df2 | All | PowerTransformer | — | 78.31 | 58.71 |
| ANN | Df3 | All | — | — | 62.14 | 46.81 |
| ANN | Df3 | All | PowerTransformer | — | 79.64 | 57.45 |
| ANN | Df4 | All | — | — | 60 | 42.55 |
| ANN | Df4 | All | StandardScaler | — | 73.6 | 51.06 |
| ANN | Df5 | All | — | — | 82.47 | 68 |
| ANN | Df5 | All | StandardScaler | — | 92.78 | 83.5 |

The corresponding recall and precision for RF, SVM, and ANN models when trained on the entire signal was calculated. The calculations were performed as follows:

$$Recall = \frac{{M}_{ii}}{{\sum }_{j} {M}_{ij}}$$

$$Precision = \frac{{M}_{ii}}{{\sum }_{j} {M}_{ji}}$$

Precision accounts for the accuracy of each class, while recall (the true positive rate) reflects the fraction of relevant results that the ML model successfully classified for the different concentrations of THC. Recall and precision can be derived from the confusion matrix. Only diagonal values of the confusion matrix (Mii) will be non-zero. The results are shown in Tables 11 and 12 below.

TABLE 11
Precision values for RF and SVM when trained
on the entire dataset for m-Z-THC

| Class | RF | | SVM | | ANN | |
|---|---|---|---|---|---|---|
| (ng/mL) | Training | Testing | Training | Testing | Training | Testing |
| 0 | 0.96 | 0.82 | 0.90 | 0.80 | 0.86 | 0.5 |
| 2 | 0.96 | 0.88 | 0.92 | 0.82 | 0.82 | 0.79 |
| 5 | 0.94 | 0.88 | 0.88 | 0.80 | 0.83 | 0.82 |

TABLE 12
Recall values for RF and SVM when trained
on the entire dataset for m-Z-THC

| Class | RF | | SVM | | ANN | |
|---|---|---|---|---|---|---|
| (ng/mL) | Training | Testing | Training | Testing | Training | Testing |
| 0 | 0.96 | 0.91 | 0.93 | 0.90 | 0.85 | 0.90 |
| 2 | 0.93 | 0.83 | 0.87 | 0.77 | 0.82 | 0.75 |
| 5 | 0.98 | 0.85 | 0.90 | 0.78 | 0.83 | 0.59 |

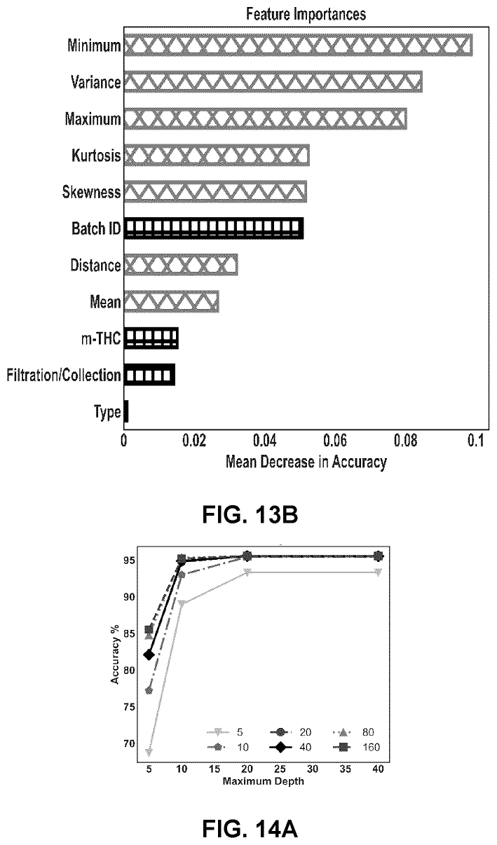

The impact of hyperparameters for RF, SVM, and ANN models was investigated. The results are shown in FIGS. 13A and 13B for the mean decrease in impurity (MDI) and the mean decrease in accuracy (MDA) respectively. The variance, minimum, maximum, kurtosis, skewness, mean, and distance were found to be the most relevant hyperparameters.

The average execution time is shown in Table 13 below.

TABLE 13
Average execution time for RF, SVM and ANN model

| Model | Computational Time (s) |
|---|---|
| RF | 0.000153 |
| SVM | 0.000086 |
| ANN | 6.957 |

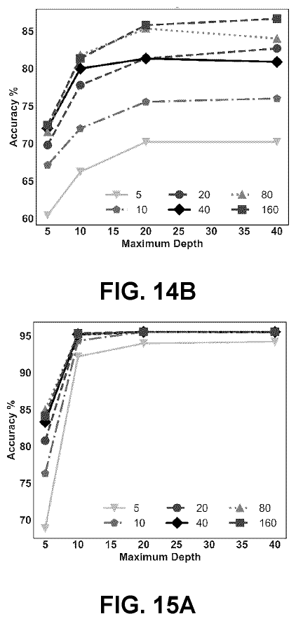

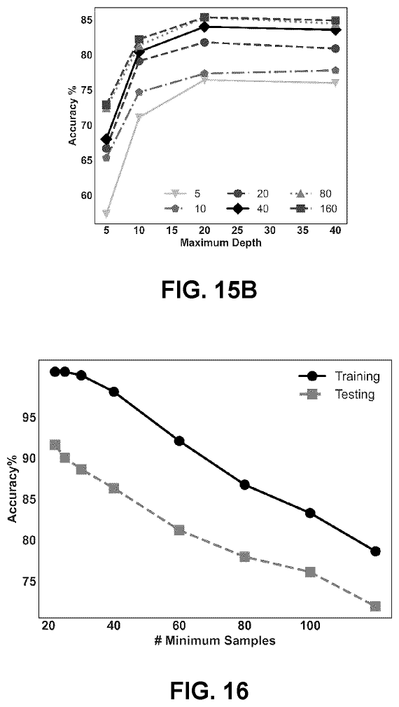

The impact of the number of trees and the maximum depth of the trees on the performance of RF models with Gini impurity is shown in FIGS. 14A-14B and with entropy is shown in FIGS. 15A-15B. In general, several parameters including the maximum depth of the tree, number of trees, and splitting criteria can have a significant impact on the performance of Random Forest models. Increasing the number of trees and the maximum depth of the trees improves the performance of the model until reaching an optimal value. The relationship between the performance of the model and its hyperparameters is not linear.

The impact of the number of minimum required samples on RF models was evaluated. The results are shown in FIG. 16. When the minimum required number of samples in each node for the splitting process increased, the accuracy of both training and testing sets dropped without significant improvement in overfitting.

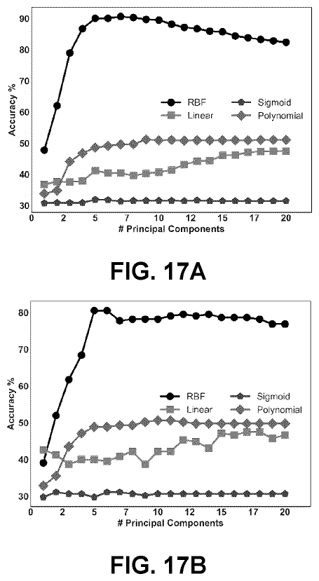

The impact of the number of principal components on different Kernel functions using the SVM model was evaluated and the results are shown in FIGS. 17A-17B and Table 14. The goal of using PCA is to reduce the dimensionality of the problem while not losing information and without changing the variance total data set. Kernel functions play a significant role in SVM models. The Radial Basis Function (RBF) has superiority over other types of kernels when the data is non-linear. On the other hand, this kernel function is computationally more expensive.

TABLE 14
Performance of RBF kernel with different
feature scaling techniques using SVM.

| # Principal | | | |
|---|---|---|---|
| Components | Feature Scaling | Training % | Testing % |
| 5 | Standard Scaler | 90 | 81 |
| | Robust Scaler | 84 | 68 |
| | MinMax Scaler | 52 | 54 |
| 6 | Standard Scaler | 90 | 81 |
| | Robust Scaler | 88 | 74 |
| | MinMax Scaler | 49 | 53 |
| 7 | Standard Scaler | 91 | 78 |
| | Robust Scaler | 90 | 77 |
| | MinMax Scaler | 48 | 51 |
| 8 | Standard Scaler | 90 | 78 |
| | Robust Scaler | 90 | 78 |
| | MinMax Scaler | 49 | 53 |
| 9 | Standard Scaler | 89 | 78 |
| | Robust Scaler | 89 | 78 |
| | MinMax Scaler | 49 | 51 |
| 10 | Standard Scaler | 89 | 78 |
| | Robust Scaler | 87 | 77 |
| | MinMax Scaler | 49 | 51 |

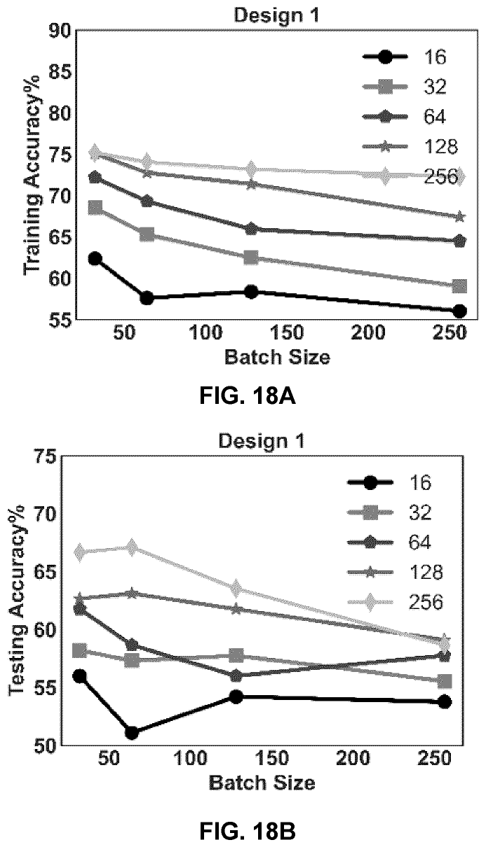

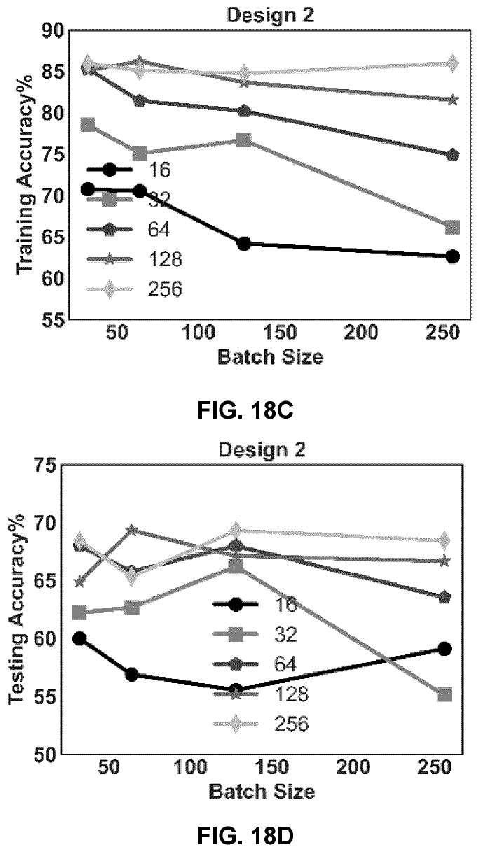

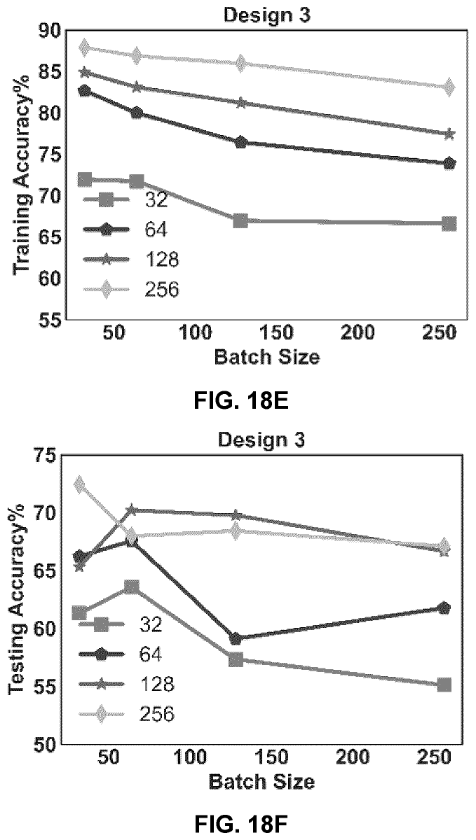

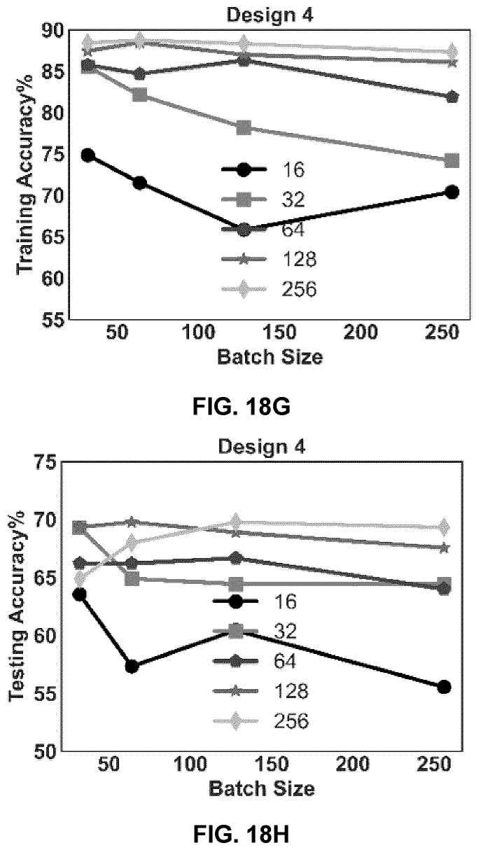

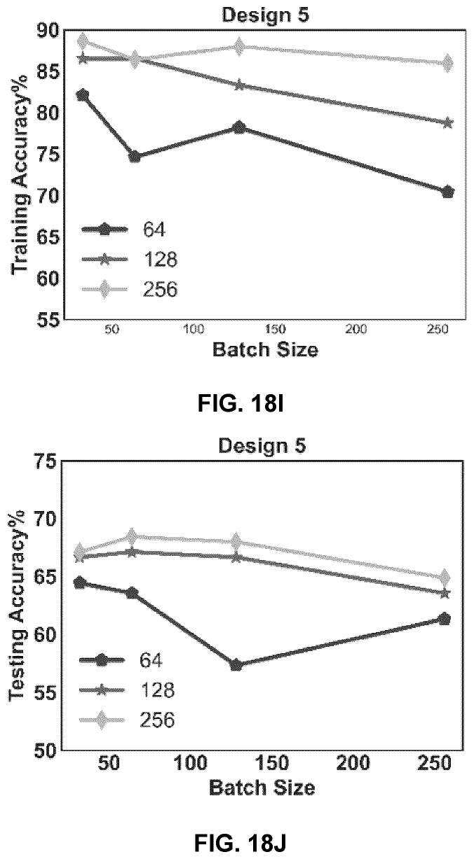

The impact of the structure of ANN was evaluated with five designs. Design 1 consisted of one hidden layer with a different number of neurons, ranging between 16 to 256. Design 2 consisted of two hidden layers with an equal number of neurons, ranging from 16 to 256. Design 3 consisted of two hidden layers with the number of neurons in the second layer ranging from 32 to 256 while the first layer has half the number of neurons in the second layer. Design 4 consisted of three hidden layers with an equal number of neurons in each layer, again varied from 16 to 256 in multiples of 2. Design 5 consisted of three hidden layers with the number of neurons in each consecutive layer twice as the previous one. The number of neurons in the last layer for Design 5 is between 64 to 256. The results are shown in FIGS. 18A-18J.

Finding the best architecture for ANN models of state of art involves trial and error. In general, the more complex structure improves the performance of the ML technique for training datasets but may lead to overfitting and high computational time. It is recommended to start with a simple structure first and increase the complexity of the model, i.e. increasing the number of hidden layers and gradually increasing the number of neurons in consecutive hidden layers.

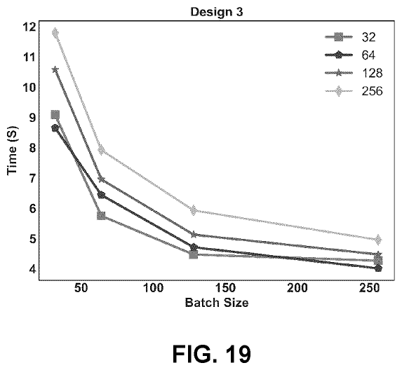

The computational time for design 3 is shown in FIG. 19. In general, an increase in the batch size often worsens the models' performance. On the other hand smaller training batches can be time-consuming.

The accuracy of training and testing for different batches was determined when training on the entire signal. The results are shown in Tables 15 and 16a.

TABLE 15
The effect of batch-to-batch variation. The RF models used the
Gini impurity criterion. The SVM models used the RBF kernel.
The fourth design architecture was used for the ANN models.

| | RF | | SVM | | ANN | |
|---|---|---|---|---|---|---|
| | Training | Testing | Training | Testing | Training | Testing |
| | Accuracy | Accuracy | Accuracy | Accuracy | Accuracy | Accuracy |
| Batch # | % | % | % | % | % | % |
| 1 | 100 | 93 | 99 | 90 | 97 | 80 |
| 2 | 100 | 100 | 95 | 89 | 96 | 76 |
| 3 | 100 | 90 | 98 | 83 | 89 | 79 |
| 4 | 100 | 92 | 99 | 84 | 93 | 84 |

TABLE 16a
Accuracy of ML techniques on different datasets trained
with the entire signal for m-Z-CBD sensors.

| | | Dimensionality | | |
|---|---|---|---|---|
| Model | Preprocessing | Reduction | Train | Test |
| RF | — | — | 100 | 64.86 |
| RF | — | PCA | 100 | 83.78 |
| SVM | StandardScaler | — | 74.82 | 62.16 |
| SVM | — | PCA | 77.55 | 62.16 |
| SVM | PowerTransformer | PCA | 91.84 | 78.37 |
| ANN | — | — | 87.76 | 67.56 |
| ANN | StandardScaler | — | 93.88 | 70.27 |

Support Vector Machine, Decision Tree, and Logistic regression were used to classify signals with and without interference for m-Z-THC and m-Z-CBD sensors. Table 16b summarizes the accuracy of each model on training and testing sets for m-Z-CBD sensor. The results demonstrated the superiority of the SVM method over other classification techniques. The entire signal features were used for training and preprocessing, and dimensionality reduction was applied on datasets before training for all methods except Decision Tree. Similar results can be observed for m-Z-THC sensor, as per Table 17.

TABLE 16b
Results of ML techniques for binary identification
of interference for m-Z-CBD Sensor.

| Technique | Training | Testing |
|---|---|---|
| Logistic Regression | 67.75 | 69.56 |
| Decision Tree | 66.12 | 63.04 |
| Support Vector | 95.63 | 92.88 |

TABLE 17
Results of ML techniques for binary identification
of interference for m-Z-THC Sensor.

| Technique | Training | Testing |
|---|---|---|
| Logistic Regression | 70.27 | 70.21 |
| Decision Tree | 70.27 | 72.34 |
| Support Vector | 95.17 | 89.92 |

The SVM model was used to classify the class of concentration of target sensor in the presence of THC/CBD. Table 18 summarizes the accuracy of results for training and testing datasets for both sensors. The results demonstrated the capability of SVM method to identify the class in the presence of interference.

TABLE 18
Results of ML techniques for multiclassification.

| Technique | Training | Testing |
|---|---|---|
| CBDi-sensor | 96 | 72 |
| THCi-Sensor | 96 | 77 |

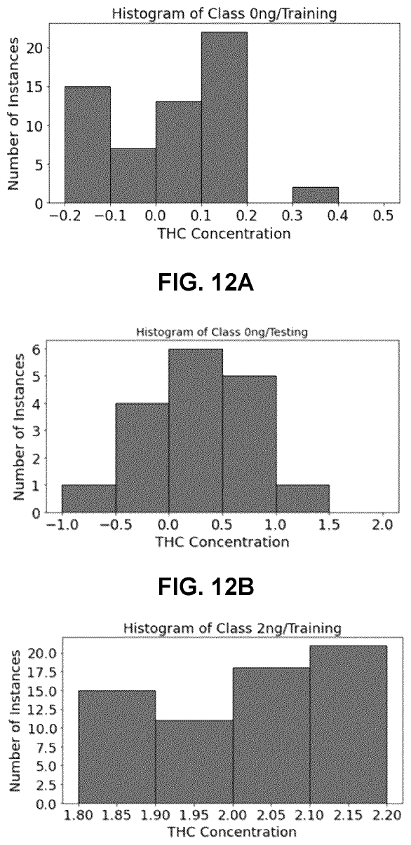

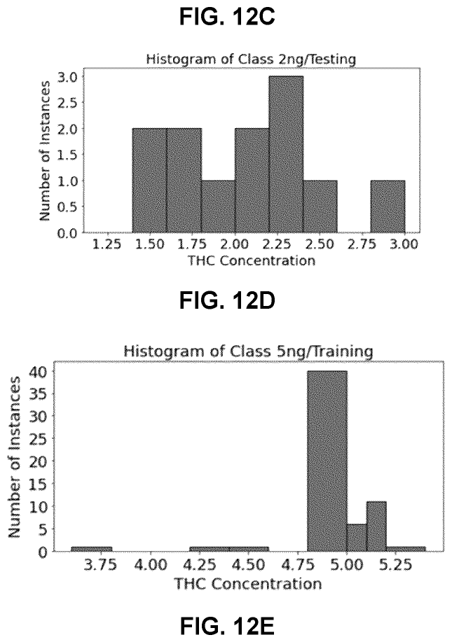

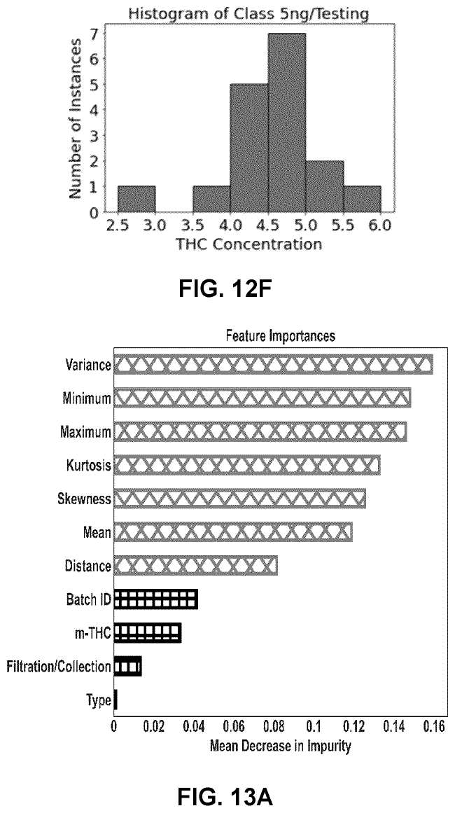

SVM regression model was deployed to predict the concentration of THC in the presence of CBD. FIGS. 12A-F illustrate the histogram of predicted results per class for training and testing sets. The result was auspicious despite SVM being trained by discrete values and not continuous concentration values.

Two supervised ML techniques, Random Forest (RF) and Support Vector Machine (SVM), were used for the classification of different concentration levels of cocaine in human saliva. RF is an ensemble ML technique that uses a bagging strategy to combine a group of decision trees (DT). Each DT is trained based on a subset of the original dataset and a subset of features. DT recursively split the training datasets into subregions based on a single feature with the lowest impurity. Contrary to DT models, RF techniques do not suffer from overfitting because of its random nature.

Electrodes were rinsed with ultrapure (Milli-Q water), and solutions were prepared using phosphate buffer saline (PBS) purchased from Sigma Aldrich as tablets. PBS solution of 0.01 M with a pH of 7.4 was used as the supporting electrolyte. Cocaine hydrochloride standard solution in methanol (1 mg/mL) was purchased from Sigma-Aldrich (Oakville, Canada). The electrochemical experiments were performed using a PalmSens™ 4 Potentiostat/Galvanostat/Impedance Analyzer connected to a computer using the PalmSens™ PSTrace Software. SPEs with carbon-based working (3 mm/0.071 cm²) and counter electrodes and silver reference were purchased from Zensor R&D (Taichung, Taiwan). Data analysis and image configuration was performed using the Origin 8.5 software.

The carbon electrodes used were thoroughly rinsed with Milli-Q water and allowed to air dry before proceeding. As a pre-treatment, 100 μL of PBS was pipetted onto the electrode and interrogated under the following square-wave voltammetry (SWV) parameters: equilibration time of 3 s, voltammetric potential scan from 0 to 1.5 V with a frequency of 15 Hz, an amplitude of 25 mV, and a step potential of 5 mV. This step was repeated three times per electrode. Next, a solution composed of PBS and methanol (9:1 ratio in volume) is prepared to be mixed with the cocaine hydrochloride as the modifying solvent. The PBS to methanol solution is mixed with cocaine hydrochloride at a 9:1 ratio in volume, referred to as the COCi solution.

This solution was then used to obtain an initial COCi deposition of 100, 150, or 200 ng, depending on how much is dispensed onto the working electrode. Once the COCi solution has been prepared, depending on the deposition required, either 1, 1.5 or 2 μL of the COCi solution is pipetted onto the working electrode. This solution was then allowed to air dry for approximately 6 minutes. Once the electrodes are dry and the solvent has been adsorbed, they are subjected to cyclic voltammetry (CV) interrogation with different concentrations of cocaine hydrochloride in saliva/PBS ranging from 0 to 100 ng/mL. The samples were prepared using a serial dilution method to ensure the difference in concentration was as accurate as possible. 65/100 μL of the samples were individually pipetted onto the electrode to cover the entire area. The parameters of CV were as follows: equilibration time of 5 s, voltammetric potential scan from 0 to 1.5 V, and a scan rate of 0.1 V/s.

The objective of SVM models is to divide classes with the most possible margin from a hyperplane. Support vectors are the nearest points to the hyperplane's margin in each class. These outlier points determine the position and orientation of the hyperplane. Finding the hyperplane often requires transforming data from its original dimension into a higher-dimension space. Kernel functions facilitate these transformations based on the similarity and distances between two data points in their original dimension.

The following tables show the results of ternary and quaternary classification for both RF and SVM applications in terms of confusion matrices. The overall testing accuracies for the binary classification were 83% and 79% for RF and SVM, respectively. Nonetheless, the testing accuracy decreased by 10-16%.

TABLE 19
Ternary Classification - Random Forest where C0, C10, and C >=25
represent the concentrations of COC in saliva (0, 10, 25 ng/mL)

| P | Training | | | Testing | | |
|---|---|---|---|---|---|---|
| A | C0 | C10 | C >=25 | C0 | C10 | C >=25 |
| C0 | 33 | 0 | 0 | 5 | 0 | 0 |
| C10 | 0 | 31 | 0 | 1 | 7 | 0 |
| C >=25 | 0 | 0 | 32 | 1 | 2 | 8 |

TABLE 20
Ternary Classification - Support Vector Machine where
C0, C10, and C >=25 represent the concentrations
of COC in saliva (0, 10, 25 ng/mL)

P

| | Training | | | Testing | | |
|---|---|---|---|---|---|---|
| A | C0 | C10 | C >=25 | C0 | C10 | C >=25 |
| C0 | 33 | 0 | 0 | 7 | 0 | 0 |
| C10 | 0 | 31 | 0 | 0 | 6 | 2 |
| C >=25 | 0 | 0 | 32 | 0 | 3 | 6 |

TABLE 21
Quaternary Classification: Random Forest where C0,
C10, C25, and C >=50 represent the concentrations
of COC in saliva (0, 10, 25, 50 ng/mL)

| | P | | | | | | | |
|---|---|---|---|---|---|---|---|---|
| | Training | | | | Testing | | | |
| A | C0 | C10 | C25 | C >=50 | C0 | C10 | C >=25 | C >=50 |
| C0 | 31 | 0 | 0 | 0 | 8 | 0 | 0 | 0 |
| C10 | 0 | 34 | 0 | 0 | 0 | 6 | 0 | 2 |
| C25 | 0 | 0 | 30 | 0 | 0 | 0 | 3 | 0 |
| C >=50 | 0 | 0 | 0 | 33 | 1 | 0 | 7 | 5 |

TABLE 22
Quaternary Classification: Support Vector Machine
where C0, C10, C25, and C >=50 represent the
concentrations of COC in saliva (0, 10, 25, 50 ng/mL)

| | P | | | | | | | |
|---|---|---|---|---|---|---|---|---|
| | Training | | | | Testing | | | |
| A | C0 | C10 | C25 | C >=50 | C0 | C10 | C >=25 | C >=50 |
| C0 | 31 | 0 | 0 | 0 | 8 | 0 | 0 | 0 |
| C10 | 0 | 34 | 0 | 0 | 1 | 4 | 1 | 1 |
| C25 | 0 | 0 | 30 | 0 | 0 | 1 | 5 | 0 |
| C >=50 | 0 | 0 | 0 | 33 | 0 | 1 | 4 | 6 |

The present Example accordingly demonstrated the use of ML in the detection of THC, CBD, and cocaine with an electrochemical sensor in saliva samples. To summarize, inaccuracies due to person-to-person saliva variations, electrode batches discrepancies, and interferences of cannabidiol were observed after the analysis of the traditional concentration vs. current responses. ML algorithms were successfully introduced to analyze the datasets to overcome these setbacks. Overall, the classification of THC samples with 0, 2, and 5 ng/mL presented accuracies between 85% and 92% for testing. In addition, the results showed the capability of ML techniques to classify and predict THC concentration in the presence of CBD interference.

The embodiments described in this document provide non-limiting examples of possible implementations of the present technology. Upon review of the present disclosure, a person of ordinary skilled in the art will recognize that changes may be made to the embodiments described herein without departing from the scope of the present technology. Yet further modifications could be implemented by a person of ordinary skill in the art in view of the present disclosure, which modifications would be within the scope of the present technology.

1. A method of sensing an analyte in a sample by electrochemical detection, the method comprising:
receiving the sample on a sample receiving region of an electrochemical sensor, the sample receiving region being in fluid communication with a sensing electrode of the sensor;
applying an electric potential scan in a target range of electric potentials to the sensing electrode to induce an electrochemical reaction with the analyte;
measuring an electrical signal from the sensing electrode while the electric potential is applied;
inputting the electrical signal into a processing device having at least one machine learning algorithm operating therein; and
executing, by the processing device, the at least one machine learning algorithm to determine from the electrical signal a presence or an absence of the analyte in the sample.

2. The method of claim 1, wherein the sensing electrode has a plurality of sensor analytes associated therewith.

3. The method of claim 1, wherein the electrochemical reaction is an oxidation, a reduction, or an enzymatic reaction.

4. The method of claim 1, wherein the electrical signal is an electric current.

5. The method of claim 1, wherein the sample comprises a body fluid, cells from a subject or a biomolecule from the subject.

6. The method of claim 5, wherein the sample comprises one or more of oral fluid, sputum, urine, tears, blood, plasma, nasal fluid, sweat, cerebral spinal fluid, suspended cells, and feces.

7. The method of claim 1, wherein the electric potential is applied using a voltammetric technique selected from square wave voltammetry, cyclic voltammetry, linear sweep voltammetry, and differential pulse voltammetry.

8. (canceled)

9. (canceled)

10. The method of claim 1, wherein the target range of electric potential is from 0 to 5 V.

11. The method of claim 1, wherein the at least one machine learning algorithm is executed by the processing device to determine the presence or the absence of the analyte in the sample comprising determining a range of values of a concentration of the analyte in the sample or determining a single value of a concentration of the analyte in the sample.

12. (canceled)

13. The method of claim 1, wherein the at least one machine learning algorithm is executed by the processing device to determine the presence or the absence of the analyte in the sample comprising determining whether the concentration of the analyte in the sample is above or below a predetermined concentration threshold.

14. The method of claim 1, wherein the at least one machine learning algorithm is configured to decrease noise present in the electrical signal for determining the presence or the absence of the analyte in the sample, the noise resulting from at least one of subject-to-subject variations in the sample, discrepancies between batches of the sensing electrode, and analog compound interference in the sample.

15. The method of claim 1, wherein the at least one machine learning algorithm is trained with at least one statistical feature of the electrical signal, the at least one statistical feature comprising at least one of a maximum, a minimum, a distance between the maximum and the minimum, a mean, a variance, a skewness, and a kurtosis.

16. The method of claim 1, wherein the at least one machine learning algorithm is trained with an entirety of the electrical signal.

17. The method of claim 16, further comprising, prior to executing the at least one machine learning algorithm, reducing a dimensionality of the electrical signal using one of principal component analysis (PCA), locally linear embedding (LLE), multidimensional scaling (MDS), t-distributed stochastic neighbor embedding (t-SNE), and linear discriminant analysis (LDA).

18. (canceled)

19. The method of claim 1, wherein the at least one machine learning algorithm is a supervised machine learning algorithm or an unsupervised machine learning algorithm.

20. The method of claim 1, wherein the at least one machine learning algorithm is configured to perform at least one of a regression analysis and a classification task to determine the concentration of the analyte from the electrical signal.

21. The method of claim 20, wherein the at least one machine learning algorithm is configured to perform the classification task using one of logistic regression, soft Regression, decision Tree, random forest (RF), and an artificial neural network (ANN).

22. The method of claim 21, wherein the at least one machine learning algorithm is configured to perform the regression analysis using one of linear regression, gradient descent, polynomial regression, regularized linear model, ridge regression, lasso regression, and support vector machine (SVM).

23. The method of claim 1, wherein the at least one machine learning algorithm comprises a plurality of different machine learning algorithms combined into an ensemble machine learning model.

24. The method of claim 1, wherein the analyte is a metabolite, a drug of abuse, or a hormone.
